(19) United States
(12) Patent Application Publication
(10) Pub. No.: US 2024/0156802 A1
(43) Pub. Date: May 16, 2024

(54) METHODS FOR TREATING DISORDERS AMELIORATED BY MUSCARINIC RECEPTOR ACTIVATION

(71) Applicant: KARUNA THERAPEUTICS, INC., Boston, MA (US)

(72) Inventors: Andrew MILLER, East Walpole, MA (US); Stephen BRANNAN, Boston, MA (US); Inderjit KAUL, Boston, MA (US); Ronald N. MARCUS, Boston, MA (US); David S. SMALL, Boston, MA (US)

(21) Appl. No.: 18/454,177

(22) Filed: Aug. 23, 2023

Related U.S. Application Data
(63) Continuation of application No. PCT/US2022/017485, filed on Feb. 23, 2022.
(60) Provisional application No. 63/213,998, filed on Jun. 23, 2021.
(60) Provisional application No. 63/153,095, filed on Feb. 24, 2021.

(51) Int. Cl. A61K 31/46; A61K 9/16; A61K 31/4439; A61P 25/18; A61P 25/28

(52) U.S. Cl. CPC A61K 31/46; A61K 9/1652; A61K 31/4439; A61P 25/18; A61P 25/28

(57) ABSTRACT

Provided herein are methods of treating a disorder ameliorated by activating muscarinic receptors in a patient 55 years or older in need thereof. The method comprises administering a total daily dose of between 25 and 200 mg xanomeline and/or a salt thereof and between 5 and 30 mg of a salt of trospium to the patient, such as a total daily dose of between 100 and 200 mg xanomeline and/or a salt thereof and between 20 and 30 mg of a salt of trospium. The present disclosure also provides a method for treating dementia-related psychosis and a method decreasing the risk of relapse in a patient having dementia-related psychosis.

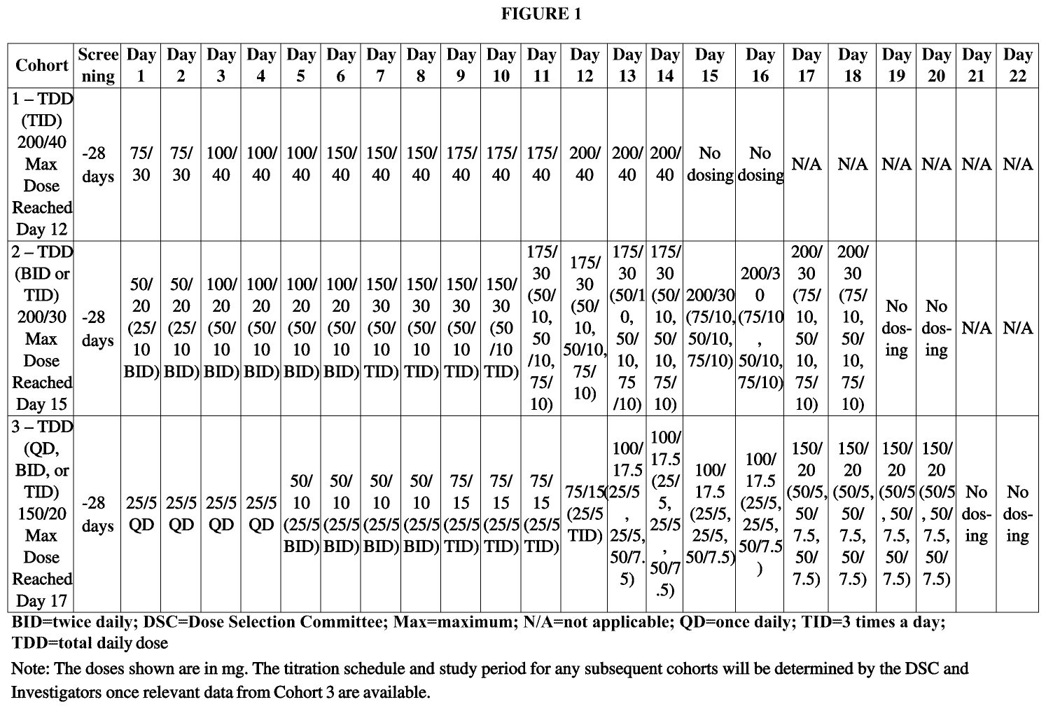

This application is a bypass continuation of International Application No. PCT/US2022/017485, filed Feb. 23, 2022, which claims priority to, and the benefit of, U.S. Provisional Patent Application Ser. No. 63/213,998 filed Jun. 23, 2021, and also claims the benefit of priority of the U.S. Provisional Patent Application Ser. No. 63/153,095 filed Feb. 24, 2021, the entireties of each are incorporated herein by reference as if written in their entireties.

The present disclosure relates to compositions and their application as pharmaceuticals for treating disorders ameliorated by activating muscarinic receptors in a human or animal subject, such as in an elderly subject, or to treat dementia-related psychosis.

Dementia-related psychosis (DRP) is a common symptom in people with dementia, whether due to Alzheimer's disease, Lewy body dementia, vascular dementia, dementia-related to Parkinson's disease, frontotemporal dementia, other forms of dementia, or related disorders. DRP refers to behaviors that can include hallucinations, delusional thinking, agitation, or aggressive behavior. These patients may have visual, auditory, and olfactory hallucinations—seeing, hearing, and smelling things that are not there—or paranoid delusions, such as suspecting a caregiver of wanting to harm them. While psychosis can be more common as dementia advances, visual hallucinations can be an early symptom of Lewy body dementia and Parkinson's-related dementia.

Atypical antipsychotics, such as risperidone, olanzapine, quetiapine, and aripiprazole, are currently the first-line agents in patients with psychotic symptoms of dementia. Divalproex (Depakote™) or carbamazepine (Tegretol™) are recommended as second-line agents in patients with inadequate response to antipsychotic agents. Nevertheless, these drugs exert detrimental effects and provide limited efficacy, especially in elderly patients who are particularly sensitive to severe adverse reactions induced by atypical antipsychotics, such as excessive sedation, orthostatic hypotension, and related complications such as falls, extrapyramidal symptoms, cognitive slowing, cardiovascular complications, and anticholinergic side effects.

These drugs were approved specifically for treating schizophrenia, which affects mostly younger adults with neurobiological deficits distinct from the behavioral and psychological symptoms of dementia. Aging induces changes in the quality and quantity of neurotransmitters, which may account for the onset of behavioral symptoms in patients with dementia. Fluctuations of neurochemicals initiate changes in the expression of certain receptors that should be targeted with specific medications, such as the muscarinic system.

Activating the muscarinic system through muscarinic agonists may treat several diseases, such as dementia-related psychosis, schizophrenia, Alzheimer's disease, Parkinson's disease, depression, movement disorders, drug addiction, pain, and neurodegeneration, such as tauopathies or synucleinopathies. Muscarinic cholinergic receptors are G-protein coupled receptors with five different receptor subtypes (M1-M5), each of which is found in the CNS with different tissue distributions. M1 and M4 subtypes have been of interest as therapeutic targets for various diseases. For instance, mood stabilizers lithium and valproic acid, used for treating bipolar depression, may affect the muscarinic system, particularly through the M4 subtype receptor. Genetic evidence directly links the muscarinic system and alcohol addiction.

In a double-blind placebo-controlled trial of schizophrenic patients using xanomeline (25-75 mg TID, 75-225 mg TDD), a muscarinic cholinergic receptor agonist with preferential activity at the M1 and M4 subtype receptors, schizophrenia was alleviated. However, because xanomeline is also bound to muscarinic receptors outside the brain, it has many serious side effects, including GI side effects, cardiac side effects, and hypersalivation. Dose-limited adverse events were problematic and led to very high discontinuation rates (including a 56% dropout rate in a 26-week study of Alzheimer's disease) and eventually to discontinuation of xanomeline development. Despite the early promise, xanomeline development halted for more than 15 years. Many companies attempted and failed to develop muscarinic receptor agonists for CNS disorders, which avoided these unacceptable side effects, but no such agonist has reached the market. Past development efforts focused on medicinal chemistry to develop molecules that would be more tolerable, typically selecting the M1 and M4 subtypes over the M2 and M3 muscarinic receptor subtypes. However, M1 and M4 activation outside the brain may still cause muscarinic-related intolerance. Very little progress has been made to mitigate adverse effects due to the activation of peripheral muscarinic receptors.

There remains a need in the art for a pharmaceutical composition with increased tolerability for xanomeline, especially to treat cognitive and psychotic disorders. The following embodiments and aspects thereof are described and illustrated with compositions and methods, which are meant to be exemplary and illustrative, not limiting in scope. In various embodiments, one or more of the above-described problems have been reduced or eliminated, while other embodiments are directed to other improvements.

### SUMMARY

Provided herein is a method of treating a disorder ameliorated by activating muscarinic receptors in an elderly patient in need thereof, the method comprising administering a total daily dose of between 25 and 200 mg xanomeline and/or a salt thereof and between 5 and 40 mg of a salt of trospium to the patient.

Also provided herein is a method of treating a dementia-related psychosis in a patient in need thereof, the method comprising administering a total daily dose of between 25 and 200 mg xanomeline and/or a salt thereof and between 5 and 40 mg of a salt of trospium to the patient.

The present disclosure further provides a method of decreasing the risk of relapse in a patient having dementia-related psychosis, comprising administering a total daily dose of between 25 and 250 mg xanomeline and/or a salt thereof and between 5 and 60 mg of a salt of trospium to the patient. In certain embodiments, when the patient is 55 years or older, the total daily dose is between 25 and 200 mg xanomeline and/or a salt thereof and between 5 and 30 mg of the salt of trospium. In certain embodiments, when the patient is younger than 55 years, the total daily dose is between 100 and 250 mg xanomeline and/or a salt thereof and between 40 and 60 mg of the salt of trospium.

Further aspects and advantages will be apparent to those of ordinary skill in the art from a review of the following detailed description. While the dosage form, method of making, and treatment method are susceptible of embodiments in various forms, the description hereafter includes specific embodiments to understand that the disclosure is illustrative and is not intended to limit the disclosure to the specific embodiments described herein.

### BRIEF DESCRIPTION OF THE DRAWINGS

The disclosure will be readily understood by the following detailed description in conjunction with the accompanying drawings, wherein like reference numerals designate like structural elements. The drawings provide exemplary embodiments or aspects of the disclosure and do not limit the scope of the disclosure.

The FIGURE shows the titration schedule for the Phase Ib clinical trial of Example 1. The doses shown are in mg. The titration schedule and study period for any subsequent cohorts were determined by the DSC and Investigators once relevant data from the previous cohort were available. BID=twice daily; DSC=Dose Selection Committee; Max=maximum; N/A=not applicable; QD=once daily; TID=3 times a day; TDD=total daily dose.

### DETAILED DESCRIPTION

Earlier development of xanomeline, a muscarinic receptor agonist, was halted due to peripheral cholinergic side effects. The current disclosure provides a dosage form with dissolution kinetics having a more effective therapeutic effect for both active ingredients, enhanced pharmacokinetics for trospium chloride, and greater dosing compliance. The current disclosure also provides dosage forms with different strengths or different ratios of the two actives.

Provided herein are the following specific embodiments. The present invention(s) is not to be limited in scope by the specific embodiments described herein, which are intended for the purpose of exemplification only. Functionally equivalent products, compositions, and methods are clearly within the scope of the invention(s), as described herein.

Embodiment 1. A method of treating a disorder ameliorated by activating muscarinic receptors in an elderly patient in need thereof, the method comprising administering a total daily dose of between 25 and 200 mg xanomeline and/or a salt thereof and between 5 and 30 mg of a salt of trospium to the patient.

Embodiment 2. The method of Embodiment 1, wherein the disorder is chosen from dementia-related psychosis, schizophrenia, Alzheimer's disease, Parkinson's disease, depression, movement disorders, pain, drug addiction, tauopathy, and synucleinopathy.

Embodiment 3. The method of Embodiment 2, wherein the disorder is dementia-related psychosis.

Embodiment 4. The method of any Embodiment, wherein the elderly patient is over 75 years old.

Embodiment 5. The method of any Embodiment, wherein the administration is oral.

Embodiment 6. The method of any Embodiment, wherein the patient is treated for at least 7 days.

Embodiment 7. The method of any Embodiment, wherein the xanomeline and/or a salt thereof is xanomeline tartrate.

Embodiment 8. The method of any Embodiment, wherein the salt of trospium is trospium chloride.

Embodiment 9. The method of any Embodiment, wherein the xanomeline and/or a salt thereof and trospium salt are administered as a pharmaceutical composition comprising a plurality of xanomeline beads having a core comprising the xanomeline or a salt thereof, and a plurality of trospium beads having a core comprising the trospium salt.

Embodiment 10. The method of any Embodiment, wherein the pharmaceutical composition is a capsule containing the plurality of xanomeline beads and the plurality of trospium beads.

Embodiment 11. The method of any Embodiment, wherein the xanomeline and/or a salt thereof is administered as a first pharmaceutical composition comprising a plurality of xanomeline beads having a core comprising the xanomeline or a salt thereof, and the salt of trospium is administered as a second pharmaceutical composition comprising a plurality of trospium beads having a core comprising the trospium salt.

Embodiment 12. The method of any Embodiment, wherein the first and second pharmaceutical compositions are administered simultaneously.

Embodiment 13. The method of any Embodiment, wherein the first pharmaceutical composition is the first capsule containing the plural of xanomeline beads, and the second pharmaceutical composition is a second capsule containing the plurality of trospium beads.

Embodiment 14. The method of any Embodiment, wherein the first and second pharmaceutical compositions are subject to different dosing schedules.

Embodiment 15. The method of any Embodiment, further comprising orally administering to the patient an increased dose of the salt of trospium and an increased dose of xanomeline and/or the salt thereof, wherein the increased dose of the salt of trospium is greater than the initial dose of the salt of trospium, and wherein the increased dose of the xanomeline and/or the salt thereof is greater than the initial dose of the xanomeline and/or the salt thereof.

Embodiment 16. The method of any Embodiment, wherein the total daily dose is chosen from
25 mg xanomeline and/or the salt thereof and 5 mg a salt of trospium,
50 mg xanomeline and/or the salt thereof and 10 mg a salt of trospium,
50 mg xanomeline and/or the salt thereof and 20 mg a salt of trospium,
75 mg xanomeline and/or the salt thereof and 15 mg a salt of trospium,
75 mg xanomeline and/or the salt thereof and 30 mg a salt of trospium,
100 mg xanomeline and/or the salt thereof and 17.5 mg a salt of trospium,
100 mg xanomeline and/or the salt thereof and 20 mg a salt of trospium,
150 mg xanomeline and/or the salt thereof and 20 mg a salt of trospium,
150 mg xanomeline and/or the salt thereof and 30 mg a salt of trospium,
175 mg xanomeline and/or the salt thereof and 30 mg a salt of trospium, and
200 xanomeline and/or the salt thereof and 30 mg a salt of trospium.

Embodiment 17. The method of any Embodiment, wherein the total daily dose is between 100 and 200 mg xanomeline and/or a salt thereof and between 10 and 30 mg of a salt of trospium.

Embodiment 18. The method of any Embodiment, wherein the total daily dose is between 100 and 200 mg xanomeline and/or a salt thereof and between 20 and 30 mg of a salt of trospium.

Embodiment 19. The method of any Embodiment, wherein the total daily dose is between 100 and 200 mg xanomeline and/or a salt thereof and about 30 mg of a salt of trospium.

Embodiment 20. The method of any Embodiment, wherein the total daily dose is about 200 mg xanomeline and/or a salt thereof and about 30 mg of a salt of trospium.

Embodiment 21. The method of any Embodiment, wherein the total daily dose is administered in two or three intervals.

Embodiment 22. The method of any Embodiment, wherein a mean Cₘₐₓ of xanomeline of about 7.3±2.0 ng/mL is attained.

Embodiment 23. The method of any Embodiment, wherein a mean Cₘₐₓ of xanomeline of about 14.2±5.3 ng/mL is attained.

Embodiment 24. The method of any Embodiment, wherein the patient has an estimated globular filtration rate (eGFR) of greater than 45.

Embodiment 25. The method of any Embodiment, wherein the patient has an estimated globular filtration rate (eGFR) of greater than 60.

The articles “a” and “an” refer to one or more than one (i.e., to at least one) of the grammatical object of the article. By way of example, “an element” means one element or more than one element.

The terms “comprise” and “comprising” are inclusive, open sense, meaning that additional elements may be included.

The term “consisting” limits the elements to those specified except for impurities ordinarily associated in addition to that.

The term “consisting essentially of” limits those specified elements and those that do not materially affect the basic and novel characteristics of the material or steps.

All ranges set forth herein include all possible subsets of ranges and any combinations of such subset ranges. By default, ranges include the stated endpoints, unless stated otherwise, where a range of values is provided, each intervening value between the upper and lower limit of that range and any other stated or intervening value in that stated range is encompassed within the disclosure. The upper and lower limits of these smaller ranges may independently be included in the smaller ranges and encompassed within the disclosure, subject to any specifically excluded limit in the stated range. Where the stated range includes one or both limits, ranges excluding either or both of those included limits are also contemplated to be part of the disclosure.

The term “wt. %” is the weight percent based on the total weight, e.g., of the core, or enteric coating, or total bead, as described in context. Unless stated otherwise, the wt. % is intended to describe the weight percent based on dry weight (e.g., for a core following drying).

The term “controlled release” is defined as a prolonged-release pattern of one or more drugs, such that the drugs are released over a period. A controlled release formulation has release kinetics that results in measurable serum levels of the drug over a period longer than what would be possible following intravenous injection or following administration of an immediate release oral dosage form. Controlled release, slow-release, sustained-release, extended-release, prolonged-release, and delayed-release have the same definitions.

The term “including” means “including but not limited to.” “Including” and “including but not limited to” are used interchangeably.

The term “mammal” is known in the art. Exemplary mammals include humans, primates, bovines, porcines, canines, felines, and rodents (e.g., mice and rats).

A “patient,” “subject,” or “host” to be treated by the subject method means either a human or non-human mammal.

“Elderly” or “geriatric” refer to a person aged 65 years or older.

The term “pharmaceutically-acceptable carrier” is art-recognized. It refers to a pharmaceutically-acceptable material, composition or vehicle, such as a liquid or solid filler, diluent, excipient, solvent, or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials that may serve as pharmaceutically acceptable carriers include sugars, such as lactose, glucose, and sucrose; starches, such as corn starch and potato starch; cellulose and its derivatives, such as sodium carboxymethyl cellulose, ethylcellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

The term “pharmaceutically-acceptable salt” or “salt” is art-recognized. It refers to a salt prepared from relatively nontoxic acids or bases, including inorganic acids and bases and organic acids and bases, including, for example, those contained in compositions of the present disclosure. Suitable non-toxic acids include inorganic and organic acids such as acetic, benzenesulfonic, benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, saccharinate, succinic, sulfuric, tartaric acid, p-toluenesulfonic, hydrochloric, hydrobromic, phosphoric, and sulfuric acids and the like.

The term “treating” is art-recognized and refers to curing as well as ameliorating at least one symptom of any condition or disorder.

In jurisdictions that forbid the patenting of methods practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation consistent with laws or regulations defining patentable subject matter is intended. In jurisdictions that do not forbid the patenting of methods practiced on the human body, the “administering” of compositions includes both methods practiced on the human body and the foregoing activities.

The term “therapeutic agent” is art-recognized and refers to any chemical moiety that is a biologically, physiologically, or pharmacologically active substance acting locally or systemically in a subject. Examples of therapeutic agents, also referred to as “drugs,” are described in well-known literature references such as the Merck Index (14th edition), the Physicians' Desk Reference (64th edition), and The Pharmacological Basis of Therapeutics (12th edition). These therapeutic agents include without limitation medicaments; vitamins; mineral supplements; substances used for the treatment, prevention, diagnosis, cure, or mitigation of a disease or illness; substances that affect the structure or function of the body, or pro-drugs, which become biologically active or more active after they have been placed in a physiological environment.

As used herein, a “dose” means the measured quantity of an active agent to be taken at one time by a patient. In certain embodiments, wherein the active agent is not xanomeline free base, the quantity is the molar equivalent to the corresponding amount of xanomeline free base. For example, often a drug is packaged in a pharmaceutically acceptable salt form, for example xanomeline tartrate, and the dosage for strength refers to the mass of the molar equivalent of the corresponding free base, xanomeline. As an example, 76 mg of xanomeline tartrate is the molar equivalent of 50 mg of xanomeline free base. Likewise, in certain embodiments, wherein the active agent is a trospium salt that is not trospium chloride, the quantity of the trospium salt is the molar equivalent to the corresponding amount of trospium chloride.

The term “psychotherapy” refers to non-pharmacological therapies. Those skilled in the art use various techniques involving verbal and other interactions with a patient to affect a positive therapeutic outcome. Such techniques include, but are not limited to, behavior therapy, cognitive therapy, psychodynamic therapy, psychoanalytic therapy, group therapy, family counseling, art therapy, music therapy, vocational therapy, humanistic therapy, existential therapy, transpersonal therapy, client-centered therapy (also called person-centered therapy), Gestalt therapy, biofeedback therapy, rational emotive behavioral therapy, reality therapy, response-based therapy, Sandplay therapy, status dynamics therapy, hypnosis, and validation therapy. Psychotherapy may involve combining two or more techniques. A therapist can select and adjust the techniques based on the individual patient's needs and responses.

The term “muscarinic disorder” refers to any disease or condition ameliorated by activating the muscarinic system. Such diseases include ones in which direct activation of muscarinic receptors themselves or inhibition of cholinesterase enzymes has produced a therapeutic effect.

The terms “diseases related to schizophrenia” and “disorders related to schizophrenia” include, but are not limited to, schizo-affective disorder, psychosis, including acute psychosis, delusional disorders, psychosis associated with Alzheimer's disease, psychosis associated with Parkinson's disease, psychotic depression, bipolar disorder, bipolar with psychosis, Huntington's disease, Lewy Body dementia, or any other disease with psychotic features.

“Psychosis” refers to an abnormal condition of the mind that results in difficulties determining what is real and not. Symptoms of psychosis include, but are not limited to, false beliefs (delusions), seeing or hearing things that others do not see or hear (hallucinations), incoherent speech, behavior that is inappropriate for the situation, sleep problems, social withdrawal, lack of motivation, and difficulties carrying out daily activities.

“Acute psychosis” refers to the quick or strong onset of psychotic symptoms in a patient, for example, as defined in “Acute and Transient Psychotic Disorder” (International Classification of Diseases-10) and “Brief Psychosis” (DSM-IV). A sharp striking delusion with quick changes in the structure occurs in the individual who has acute psychosis after a short preliminary period of anxiety, insomnia, and confusion. Acute psychosis can include acute psychotic exacerbation when a patient may respond to hallucinations or delusions. Acute psychosis lasts for a short time, typically from one to two weeks.

The term “activator” means a molecule described as an agonist, partial agonist, co-agonist, physiological agonist, potentiator, stimulator, allosteric potentiator, positive allosteric modulator, allosteric agonist, or a molecule that increases the activity or signaling of receptors directly or indirectly.

The term “inhibitor” means a molecule described as an antagonist, partial antagonist, competitive antagonist, non-competitive antagonist, uncompetitive antagonist, silent antagonist, inverse agonist, reversible antagonist, physiological antagonist, irreversible antagonist, inhibitor, reversible inhibitor, irreversible inhibitor, negative allosteric modulator, allosteric antagonist, or a molecule that decreases the activity or signaling of receptors directly or indirectly.

As used herein, an “adverse event” is any untoward medical occurrence associated with treatment with a pharmaceutical composition described herein. A “mild adverse event” is easily tolerated by the subject, causes minimal discomfort, and does not interfere with everyday activities. A “moderate adverse event” is sufficiently discomforting to interfere with everyday activities; intervention may be needed. A “severe adverse event” prevents everyday activities; treatment or other intervention is usually needed. A “serious adverse event” results in death; is life-threatening (immediate risk of death from the event as it occurred); requires or prolongs inpatient hospitalization; results in persistent or significant disability/incapacity; or results in a congenital anomaly/disability, cancer, or drug overdose. An adverse event is incapacitating or disabling if it results in a substantial or permanent disruption of the subject's ability to carry out normal life functions.

As used herein, a patient is said to “tolerate” a dose of a compound if administering that dose to that patient does not result in an unacceptable adverse event or an unacceptable combination of adverse events. One of skill in the art will appreciate that tolerance is a subjective measure and that what may be tolerable to one patient may not be tolerable to a different patient. For example, one patient may not be able to tolerate a headache. In contrast, a second patient may find headaches tolerable but is not able to tolerate vomiting. For a third patient, either headache alone or vomiting alone is tolerable. Still, the patient cannot tolerate the combination of headache and vomiting, even if the severity of each is less than when experienced alone.

The term “maximum tolerated dose” means the highest dose of a drug or therapeutic that a patient can take without the patient experiencing intolerable side effects. The maximum tolerated dose is typically determined empirically in clinical trials.

The term “muscarinic receptors” refers to G-protein linked receptors that bind the neurotransmitter acetylcholine. To date, five subtypes of the muscarinic receptor have been identified. “M1” means the subtype one muscarinic receptor. “M2” means the subtype two muscarinic receptor. “M3” means the subtype three muscarinic receptor. “M4” means the subtype four muscarinic receptor. “M5” means the subtype five muscarinic receptor.

The term “antipsychotic” refers to a drug that diminishes psychosis, hallucinations, or delusions. Antipsychotics include, but are not limited to haloperidol, droperidol, chlorpromazine, fluphenazine, perphenazine, prochlorperazine, thioridazine, trifluoperazine, mesoridazine, periciazine, promazine, triflupromazine, levomepromazine, promethazine, pimozide, chlorprothixene, flupenthixol, thiothixene, zuclopenthixol, clozapine, olanzapine, risperidone, quetiapine, ziprasidone, amisulpride, asenapine, paliperidone, zotepine, aripiprazole, bifeprunox, and tetrabenazine.

The term “anxiolytics” refers to drugs that reduce anxiety, fear, panic, or related feelings. Such drugs include, but are not limited to, benzodiazepines (e.g., alprazolam, chlordiazepoxide, clonazepam, clorazepate, diazepam, lorazepam), buspirone, barbiturates (e.g., amobarbital, pentobarbital, secobarbital, phenobarbital), and hydroxyzine.

The term “anti-depressants” refers to drugs that alleviate depression and related conditions (e.g., dysthymia). Such drugs include, but are not limited to, selective serotonin-reuptake inhibitors (SSRIs, e.g., citalopram, escitalopram, fluoxetine, fluvoxamine, paroxetine, sertraline), serotonin-norepinephrine reuptake inhibitors (SNRIs, e.g., desvenlafaxine, duloxetine, milnacipran, venlafaxine), mianserin, mirtazapine, norepinephrine reuptake inhibitors (e.g., atomoxetine, mazindol, reboxetine, viloxazine), bupropion, tianeptine, agomelatine, tricyclic antidepressants (e.g., amitriptyline, clomipramine, doxepin, imipramine, trimipramine, desipramine, nortriptyline, protriptyline), and monoamine oxidase inhibitors (e.g., isocarboxazid, moclobemide, phenelzine, selegiline, tranylcypromine).

The terms “sedatives” or “tranquilizers” refer to drugs that induce somnolence, promote a feeling of being tired or desire to sleep, or promote a state of unconsciousness. Such drugs include, but are not limited to, benzodiazepines, barbiturates (e.g., amobarbital, pentobarbital, secobarbital, phenobarbital), eszopiclone, zaleplon, zolpidem, and zopiclone.

“Mini-Mental State Examination” (MMSE) is a brief 30-point questionnaire used to quantitatively assess cognition. The MMSE includes simple questions and problems in a number of areas: the time and place of testing, repeating lists of words, arithmetic, language use, and comprehension, and copying a drawing.

Neuropsychiatric Inventory Clinician (NPI-C) is a valid and reliable clinician-administered scale used to assess the occurrence, severity, and meaningful change in neuropsychiatric symptoms specific to those experienced by people with dementia. It has fourteen domains: delusions, hallucinations, agitation, aggression, dysphoria, anxiety, elation/euphoria, apathy/indifference, disinhibition, irritability/lability, aberrant motor disturbances, sleep disorders, appetite and eating disorders, and aberrant vocalizations.

A clinical impression rating is assigned to each NPI-C item for the specific domain. The clinician rater assigns the rating incorporating all sources of information available (caregiver interview, patient interview, patient data, other relevant information) and is focused on the previous four-week timeframe. The clinical impression is rated on 0 to 3 point scale; 0=none; 1=mild: produces little stress; 2=moderate: distressing and causes substantial behavioral abnormalities; 3=marked: a major source of behavioral abnormality.

The NPI-C Core scale includes four domains from the NPI-C scale, namely, delusions, hallucinations, agitation, and aggression. These four domains include the following number of items to be rated by the clinician: delusions, 8 items (maximum score=24); hallucinations, 7 items (maximum score=21); agitation, 13 items (maximum score=39); and aggression, 8 items (maximum score=24). The maximum score for the NPI-C Core scale is 108. In certain embodiments, the NPI-C assessment is performed before all the other scale assessments at all visits at which it occurs. The four domains of NPI-C may be analyzed together or independently, or in any combination thereof, to assess potential benefits to patients. In particular, the NPIC-C hallucinations and delusions domains is particularly relevant when assessing potential benefits on psychosis.

### Method of Treating

Provided is a method of treating a disorder ameliorated by activating muscarinic receptors in a patient in need thereof aged 55 years or older. Also provided is a method of treating dementia-related psychosis in a patient in need thereof, such as a patient aged 55 years or older, for example, 60 years or older, or 65 years or older. Also provided is a method of preventing relapse in a patient with dementia-related psychosis. In certain embodiments, the method comprises administering a total daily dose of between 25 and 200 mg xanomeline and/or a salt thereof and between 5 and 30 mg of a salt of trospium to the patient.

While activators of M1 and M4 muscarinic receptors have been suggested to be efficacious treatments for schizophrenia, the activation of muscarinic receptors located outside the brain has resulted in side effects that barred xanomeline from the clinic. For instance, in both Phase I and subsequent trials, the muscarinic agonist xanomeline had unacceptable GI and other side effects linked to the binding of muscarinic receptors in the body's periphery. Combining xanomeline with trospium chloride, the desired therapeutic effect is achieved while diminishing or eliminating the side effects of activating muscarinic receptors located outside the brain.

The tolerability of xanomeline, a muscarinic activator, is increased by co-administering trospium chloride, a muscarinic antagonist. The most common adverse events observed with administering xanomeline are nausea, vomiting, diarrhea, excessive sweating, and excessive salivation (so-called cholinergic adverse events). A common anticholinergic adverse event observed with administering trospium chloride is dry mouth (xerostomia). The disclosed compositions reduced the incidence of these adverse events in humans, evincing increased xanomeline tolerability. In certain embodiments, after at least 4 weeks of treatment, the occurrence of a cholinergic or anticholinergic adverse event is not statistically distinguishable from a placebo control. In certain embodiments, after at least 4 weeks of treatment, at least one of nausea, vomiting, and dry mouth occurs at about the same rate as an untreated patient. In certain embodiments, at least one adverse event which occurred at the start of oral administration is reduced to its pretreatment level after five weeks of treatment.

KarXT is currently being investigated for the treatment of schizophrenia. It is anticipated that it will differ from established therapies by having a different mechanism of action from current antipsychotics without directly affecting the dopamine receptor. This suggests that KarXT will not have many of the common problematic side effects of current antipsychotics (movement disorders, hyperprolactinemia, metabolic syndrome, etc.). KarXT may also be pro-cognitive, which means that it could have additional long-term benefits for schizophrenic subjects who have cognitive impairment.

In one embodiment, xanomeline combined with trospium chloride treats an animal. In a further embodiment, the animal is a mammal. In an embodiment, the mammal is a human being.

In one embodiment, trospium chloride decreases the side effects associated with xanomeline. Such side effects include, but are not limited to, GI side effects, cardiac side effects, excessive sweating, and excessive salivation. The use of trospium with xanomeline allows the xanomeline to be used clinically when the xanomeline would not otherwise be used clinically due to its side effects. In another embodiment, the use of trospium chloride with the xanomeline allows for the xanomeline to achieve a higher maximum tolerated dose than xanomeline would otherwise achieve.

Various time and resource-intensive methods demonstrated the efficacy of the combination of xanomeline and trospium chloride. For example, animal models demonstrate the efficacy of new therapeutics for schizophrenia, including pharmacological models (e.g., ketamine model) and genetic models (e.g., DISC1 mouse). Likewise, animal models, including rodents, dogs, and non-human primates, demonstrate the side effect profile of pharmacological agents. Animal models are an experimental proxy for humans but may suffer from deficiencies in the physiological differences between humans and animals and may have limited predictive power for human experiments, particularly for central nervous system disorders. Alternatively, the disclosed combination can be tried in controlled clinical trials of people. Standard measures based on patient self-report can be used by those skilled in the art to assess various side effects such as GI discomfort. As another example, objective physiological measures (e.g., EKGs) may be used by those skilled in the art. A set of standard measures has also been developed to assess schizophrenia symptoms, including the Brief Psychiatric Rating Scale (BPRS), the Positive and Negative Syndrome Scale (PANSS), and Clinical Global Impression (CGI). Typically, clinical trials are double-blinded, where one group of patients receives an inactive placebo, and the other group the active intervention.

The Positive and Negative Syndrome Scale (PANSS) is a medical scale used for measuring symptom severity of patients with schizophrenia. The name refers to the two types of symptoms in schizophrenia, as defined by the American Psychiatric Association: positive symptoms, which refer to an excess or distortion of normal functions (e.g., hallucinations and delusions), and negative symptoms, which represent a diminution or loss of normal functions. Some of these functions which may be lost include normal thoughts, actions, the ability to tell fantasies from reality, and the ability to properly express emotions.

The PANSS is a relatively brief interview of about 45 to 50 minutes. The interviewer must be trained to a standardized level of reliability. The patient is rated from 1 to 7 on 30 different symptoms in three categories based on the interview and reports of family members or primary care hospital workers. The first category of the PANSS is the positive scale, comprising 7 Items (minimum score=7, maximum score=49): delusions, conceptual disorganization, hallucinations, excitement, grandiosity, suspiciousness/persecution, and hostility. The second category is the negative scale, comprising 7 items (minimum score=7, maximum score=49): blunted affect, emotional withdrawal, poor rapport, passive/apathetic social withdrawal, difficulty in abstract thinking, lack of spontaneity, and flow of conversation, stereotyped thinking. The third category is the General Psychopathology scale, which comprises 16 items (minimum score=16, maximum score=112): somatic concern, anxiety, guilt feelings, tension, mannerisms and posturing, depression, motor retardation, uncooperativeness, unusual thought content, disorientation, poor attention, lack of judgment and insight, disturbance of volition, poor impulse control, preoccupation, and active social avoidance.

PANSS Marder factor score is the sum of five negative scales and two general scales (N1. Blunted affect; N2. Emotional withdrawal; N3. Poor rapport; N4. Passive/apathetic social withdrawal; N6. Lack of spontaneity; G7. Motor retardation; and G16. Active social avoidance). If a patient has a PANSS assessment recorded, but any of the items are missing, the last non-missing score for the individual item from previous assessments will be carried forward. If more than 30% of the items are missing at a particular visit, the respective positive score is not calculated. It is treated as missing data in the analysis.

Because 1 rather than 0 is given the lowest score for each item, a patient cannot score lower than 30 for the total PANSS score. Subscores can be given separately for the positive items, negative items, and general psychopathology. The maximum possible total score is 210. In the original publication on the PANSS scale, 101 adult patients (20-68 years old) with schizophrenia were ranked. Their mean scores were a positive scale of 18.20, a negative scale of 21.01, and general psychopathology of 37.74. The mean total PANSS score for these subjects was 76.95.

In certain embodiments, the Positive and Negative Syndrome Scale (PANSS) total score for the subject decreases by at least 10 points more than the placebo, for example, after five treatment weeks. In certain embodiments, the PANSS positive subscore decreases by at least 3 points more than the placebo, for example, after five treatment weeks. In certain embodiments, the PANSS negative subscore decreases by at least 2 points than the placebo, for example, after five treatment weeks.

In certain embodiments, the PANSS total score for the subject decreases by at least 10 points compared to baseline for the subject, for example, after five treatment weeks. In certain embodiments, the PANSS positive subscore decreases by at least 3 points compared to baseline for the subject, for example, after five treatment weeks. In certain embodiments, the PANSS negative subscore decreases by at least 2 points compared to baseline for the subject, for example, after five treatment weeks.

Another scale used to assess patients is the Clinical Global Impression-Severity scale (CGI-S). This 7-point scale requires the clinician to rate the severity of the patient's illness at the time of assessment relative to the clinician's experience with patients who have the same diagnosis. Possible ratings are (1) Normal, not at all ill; (2) Borderline mentally ill; (3) Mildly ill, (4) Moderately ill; (5) Markedly ill; (6) Severely ill, and (7) Among the most extremely ill patients. Among schizophrenia patients, changes in the CGI-S follow a consistent pattern relative to more objective PANSS scoring.

Before administering the disclosed combinations, patients may have a lead-in period from one to fourteen days, during which lead-in period trospium chloride is given alone. In one embodiment, the trospium chloride is administered for one or more dose periods before administering xanomeline to accumulate trospium chloride in the body or for the trospium chloride to reach or approach steady-state exposure levels. This accumulation, or higher exposure levels of the trospium chloride, increases the blockade of muscarinic receptors outside of the brain and reduces adverse events when xanomeline is administered. In another embodiment, the trospium chloride is administered for one or more days before xanomeline.

Before administering the disclosed combinations, patients may discontinue any prior use of antipsychotic drugs. In some embodiments, the patients will discontinue such drugs for at least one week, such as two weeks. In some embodiments, patients do not discontinue any prior use of such antipsychotic drugs, and the disclosed combinations are co-administered with such drugs.

The present disclosure further provides a method of treating acute psychosis in a patient in need thereof. The method comprises orally administering to the patient twice daily an oral pharmaceutical composition comprising xanomeline or a salt thereof, and a salt of trospium. 1. In certain embodiments, when the total daily dose is between 200 and 250 mg xanomeline and/or a salt thereof and between 40 and 60 mg of the salt of trospium, at least about an 11.6 point mean reduction in total PANNS score compared to placebo is achieved. In certain embodiments, at least a 3 point mean reduction in PANSS positive subscore compared to placebo is achieved. In certain embodiments, at least a 2 point reduction in the PANSS negative subscore compared to placebo is achieved. In certain embodiments, the reduction in the PANSS score is achieved within about 5 weeks. In certain embodiments, before administering the oral pharmaceutical composition, the patient had a CGI-S score of 4-7.

In certain embodiments, when the total daily dose is between 200 and 250 mg xanomeline and/or a salt thereof and between 40 and 60 mg of the salt of trospium, at least about an 11.6 point mean reduction in total PANSS score compared to baseline for the patient is achieved. In certain embodiments, at least a 3 point mean reduction in PANSS positive subscore compared to baseline for the patient is achieved. In certain embodiments, at least a 2 point reduction in the PANSS negative subscore compared to baseline for the patient is achieved.

In certain embodiments, the patient has a diagnosis of schizophrenia. In certain embodiments, the patient has acute psychosis. In certain embodiments, the patient has psychosis associated with Alzheimer's disease. In certain embodiments, the patient has a schizo-affective disorder. In certain embodiments, the patient has psychosis. In certain embodiments, the patient has a delusional disorder. In certain embodiments, the patient has psychosis associated with Parkinson's disease. In certain embodiments, the patient has psychotic depression. In certain embodiments, the patient has bipolar disorder. In certain embodiments, the patient has bipolar disorder with psychosis. In certain embodiments, the patient has Huntington's disease. In certain embodiments, the patient has Lewy Body dementia.

In certain embodiments, the patient previously had been administered one or more antipsychotics. In certain embodiments, the patient was an inadequate responder to such administration. In certain embodiments, the patient was treatment-resistant.

In certain embodiments, the patient is an adult. In certain embodiments, the patient is elderly, e.g., above the age of 55 years, such as above the age of 65 years. In certain embodiments, the patient has dementia-related psychosis.

### Dose Titration

In certain embodiments, the method further comprises orally administering to the patient an increased dose of the salt of trospium and an increased dose of xanomeline and/or the salt thereof, wherein the increased dose of the salt of trospium is greater than the initial dose of the salt of trospium, and wherein the increased dose of the xanomeline and/or the salt thereof is greater than the initial dose of the xanomeline and/or the salt thereof.

Treatment may be initiated with smaller dosages. After that, small increments may increase the dosage until a balance between therapeutic effect and side effects is attained. While the subject is being treated, the patient's health may be monitored by measuring one or more of the relevant indices at predetermined times during the treatment period. Treatment, including composition, amounts, administration, and formulation times, may be adjusted per such monitoring. The patient may be periodically reevaluated to determine improvement by measuring the same parameters. Adjustments to the disclosed composition administered and possibly to the administration time may be made based on these reevaluations.

Provided is a method of treating dementia-related psychosis in a patient in need thereof, the method comprising: orally administering to the patient twice daily an oral pharmaceutical composition comprising a plurality of xanomeline beads comprising xanomeline or a salt thereof, and a plurality of trospium beads comprising a salt of trospium, via a titration scheme that comprises up-titration of the xanomeline, or a salt thereof, and the salt of trospium.

Also provided is a method of treating dementia-related psychosis in a patient in need thereof, the method comprising: orally administering twice daily an oral pharmaceutical composition comprising a plurality of xanomeline beads comprising xanomeline or a salt thereof, and a plurality of trospium beads comprising a salt of trospium, via a titration scheme that comprises up-titration of the xanomeline, or a salt thereof, and the salt of trospium until an amount equivalent to 200 mg xanomeline free base and an amount equivalent to 20 mg trospium chloride is administered.

In certain embodiments, the xanomeline, or a salt thereof, is administered for the first period in a first amount, and then the first amount is increased to a second amount. In certain embodiments, the first amount of xanomeline is equivalent to 50 mg xanomeline free base. In certain embodiments, the first period for the xanomeline administration is between 1 and 5 days, such as 2 days. In certain embodiments, the second amount of xanomeline is equivalent to 100 mg xanomeline free base.

In certain embodiments, the method further comprises administering the xanomeline, or a salt thereof, for the second period in the second amount and then increasing the second amount to a third amount. In certain embodiments, the second period for xanomeline administration is between three days and a week. In certain embodiments, the third amount of xanomeline is equivalent to 125 mg xanomeline free base.

In certain embodiments, the salt of trospium is administered for the first period in a first amount, and the first amount is increased to a second amount. In certain embodiments, the first amount of the salt of trospium is equivalent to 20 mg trospium chloride. In certain embodiments, the first period for trospium administration is at least a week. In certain embodiments, the second amount of the salt of trospium is equivalent to 30 mg trospium chloride.

In certain embodiments, if the patient is not tolerating the higher dose of xanomeline, or a salt thereof, and the salt of trospium, the amount of xanomeline, or a salt thereof, and the salt of trospium administered to the patient is decreased.

In certain embodiments, the xanomeline, or a salt thereof, and the salt of trospium are administered without causing a severe adverse event.

In certain embodiments, when the patient is 55 years or older, the initial dose is a total daily dose of 60 mg xanomeline and/or a salt thereof and 6 mg the salt of trospium administered as 20 mg xanomeline and/or a salt thereof and 2 mg the salt of trospium in three doses.

In certain embodiments, when the patient is 55 years or older, after a first time period of the administration, the initial dose is increased to a total daily dose of 90 mg xanomeline and/or a salt thereof and 9 mg salt of trospium administered as 30 mg xanomeline and/or a salt thereof and 3 mg salt of trospium in three doses. In certain embodiments, the first time period is between 1 and 14 days, such as 1 day, 2 days, 3 days, 4 days, 5 days, 6 days, 7 days, 8 days, 9 days, 10 days, 11 days, 12 days, 13 days, or 14 days. In certain embodiments, the first time period is 7 days.

In certain embodiments, after a second time period of the administration, if the patient who is 55 years or older tolerates the increased dose and if the patient has had an adequate response, the increased dose is further increased to the total daily dose of 120 mg xanomeline and/or a salt thereof and 12 mg salt of trospium administered as 40 mg xanomeline and/or a salt thereof and 4 mg salt of trospium in three doses. In certain embodiments, the second time period is between 7 and 21 total days, such as 7 total days, 8 total days, 9 total days, 10 total days, 11 total days, 12 total days, 13 total days, 14 total days, 15 total days, 16 total days, 17 total days, 18 total days, 19 total days, 20 total days, and 21 total days. In certain embodiments, the second time period is 14 total days. In certain embodiments, if the patient does not tolerate the increased dose, an optimized dose is the initial dose.

In certain embodiments, after about 21 total days of the administration, if the patient who is 55 years or older tolerates the further increased dose and if the patient has had an adequate response, the further increased dose is again increased to the total daily dose of 150 mg xanomeline and/or a salt thereof and 15 mg salt of trospium administered as 50 mg xanomeline and/or a salt thereof and 5 mg salt of trospium in three doses. In certain embodiments, the third time period is between 14 and 28 total days, such as 14 total days, 15 total days, 16 total days, 17 total days, 18 total days, 19 total days, 20 total days, 21 total days, 22 total days, 23 total days, 24 total days, 25 total days, 26 total days, 27 total days, and 28 total days. In certain embodiments, the third time period is 21 total days. In certain embodiments, if the patient does not tolerate the further increased dose, an optimized dose is the increased dose.

In certain embodiments, after a fourth time period, if the patient who is 55 years or older tolerates the again increased dose and if the patient has had an adequate response, the again increased dose is increased to the total daily dose of 200 mg xanomeline and/or a salt thereof and 20 mg salt of trospium administered as 66.7 mg xanomeline and/or a salt thereof and 6.67 mg salt of trospium in three doses. In certain embodiments, the fourth time period is between 21 and 35 total days, such as 21 total days, 22 total days, 23 total days, 24 total days, 25 total days, 26 total days, 27 total days, 28 total days, 29 total days, 30 total days, 31 total days, 32 total days, 33 total days, 34 total days, and 35 total days. In certain embodiments, the fourth time period is 28 total days. In certain embodiments, if the patient does not tolerate the again increased dose, an optimized dose is the further increased dose.

In certain embodiments, when the patient is younger than 55 years, the initial dose is a total daily dose of 100 mg xanomeline and/or a salt thereof and 40 mg of the salt of trospium administered as 50 mg xanomeline and/or a salt thereof and 20 mg the salt of trospium in two doses. In certain embodiments, after a first time period, the initial dose is increased to a total daily dose of 200 mg xanomeline and/or a salt thereof and 40 mg salt of trospium administered as 100 mg xanomeline and/or a salt thereof and 20 mg salt of trospium in two doses. In certain embodiments, the first time period is between 1 and 7 days, such as 1 day, 2 days, 3 days, 4 days, 5 days, 6 days, or 7 days. In certain embodiments, the first time period is 1 or 2 days.

In certain embodiments, wherein after a second time period, if the patient who is younger than 55 years tolerates the increased dose and if the patient has had an adequate response, the increased dose is further increased to the total daily dose of 250 mg xanomeline and/or a salt thereof and 60 mg salt of trospium administered as 125 mg xanomeline and/or a salt thereof and 30 mg salt of trospium in two doses. In certain embodiments, the second time period is between 3 and 14 total days, such as 3 total days, 4 total days, 5 total days, 6 total days, 7 total days, 8 total days, 9 total days, 10 total days, 11 total days, 12 total days, 13 total days, or 14 total days. In certain embodiments, the second time period is 3-7 total days. In certain embodiments, if the patient does not tolerate the increased dose, an optimized dose is the initial dose.

“Total days” refers to the time period of administration from the initial dose. As such, the first time period is a subset of the second time period. The second time period is a subset of the third time period. And the third time period is a subset of the fourth time period.

In certain embodiments, the total daily dose is chosen from
25 mg xanomeline and/or the salt thereof and 5 mg a salt of trospium,
50 mg xanomeline and/or the salt thereof and 10 mg a salt of trospium,
50 mg xanomeline and/or the salt thereof and 20 mg a salt of trospium,
60 mg xanomeline and/or a salt thereof and 6 mg a salt of trospium,
75 mg xanomeline and/or the salt thereof and 15 mg a salt of trospium,
75 mg xanomeline and/or the salt thereof and 30 mg a salt of trospium,
90 mg xanomeline and/or a salt thereof and a 9 mg salt of trospium,
100 mg xanomeline and/or the salt thereof and 17.5 mg a salt of trospium,
100 mg xanomeline and/or the salt thereof and 20 mg a salt of trospium,
120 mg xanomeline and/or a salt thereof and 12 mg salt of trospium,
150 mg xanomeline and/or the salt thereof and 15 mg a salt of trospium,
150 mg xanomeline and/or the salt thereof and 20 mg a salt of trospium,
150 mg xanomeline and/or the salt thereof and 30 mg a salt of trospium,
175 mg xanomeline and/or the salt thereof and 30 mg a salt of trospium,
200 mg xanomeline and/or the salt thereof and 20 mg a salt of trospium, and
200 mg xanomeline and/or the salt thereof and 30 mg a salt of trospium.

In certain embodiments, the total daily dose is chosen from
60 mg xanomeline and/or a salt thereof and 6 mg a salt of trospium,
90 mg xanomeline and/or a salt thereof and a 9 mg salt of trospium,
120 mg xanomeline and/or a salt thereof and 12 mg salt of trospium,
150 mg xanomeline and/or the salt thereof and 15 mg a salt of trospium, and
200 xanomeline and/or the salt thereof and 20 mg a salt of trospium.

In certain embodiments, the total daily dose is 25 mg xanomeline and/or the salt thereof and 5 mg a salt of trospium.

In certain embodiments, the total daily dose is 50 mg xanomeline and/or the salt thereof and 10 mg a salt of trospium.

In certain embodiments, the total daily dose is 50 mg xanomeline and/or the salt thereof and 20 mg a salt of trospium.

In certain embodiments, the total daily dose is 75 mg xanomeline and/or the salt thereof and 15 mg a salt of trospium.

In certain embodiments, the total daily dose is 75 mg xanomeline and/or the salt thereof and 30 mg a salt of trospium.

In certain embodiments, the total daily dose is 100 mg xanomeline and/or the salt thereof and 17.5 mg a salt of trospium.

In certain embodiments, the total daily dose is 100 mg xanomeline and/or the salt thereof and 20 mg a salt of trospium.

In certain embodiments, the total daily dose is 100 mg xanomeline and/or the salt thereof and 40 mg a salt of trospium.

In certain embodiments, the total daily dose is 150 mg xanomeline and/or the salt thereof and 20 mg a salt of trospium.

In certain embodiments, the total daily dose is 150 mg xanomeline and/or the salt thereof and 30 mg a salt of trospium.

In certain embodiments, the total daily dose is 150 mg xanomeline and/or the salt thereof and 40 mg a salt of trospium.

In certain embodiments, the total daily dose is 175 mg xanomeline and/or the salt thereof and 30 mg a salt of trospium.

In certain embodiments, the total daily dose is 175 mg xanomeline and/or the salt thereof and 40 mg a salt of trospium.

In certain embodiments, the total daily dose is 200 mg xanomeline and/or the salt thereof and 30 mg a salt of trospium.

In certain embodiments, the total daily dose is 200 mg xanomeline and/or the salt thereof and 40 mg a salt of trospium.

In one embodiment, xanomeline and trospium chloride are administered to a patient 6 times during a 24-hour period. In another embodiment, xanomeline and trospium chloride are administered to a patient 5 times during a 24-hour period. In another embodiment, xanomeline and trospium chloride are administered to a patient 4 times during a 24-hour period. In an embodiment, xanomeline and trospium chloride are administered to a patient 3 times during a 24-hour period. In another embodiment, xanomeline and trospium chloride are administered to a patient twice during a 24-hour period. In another embodiment, xanomeline and trospium chloride are administered to a patient once during a 24-hour period.

In certain embodiments, the administration comprises a dosing schedule of 25 mg xanomeline and/or the salt thereof and 5 mg a salt of trospium twice daily.

In certain embodiments, the administration comprises a dosing schedule of 25 mg xanomeline and/or the salt thereof and 5 mg a salt of trospium thrice daily.

In certain embodiments, the administration comprises a dosing schedule of 25 mg xanomeline and/or the salt thereof and 5 mg a salt of trospium twice daily, and 50 mg xanomeline and/or the salt thereof and 7.5 mg a salt of trospium once daily.

In certain embodiments, the administration comprises a dosing schedule of 25 mg xanomeline and/or the salt thereof and 10 mg a salt of trospium twice daily.

In certain embodiments, the administration comprises a dosing schedule of 50 mg xanomeline and/or the salt thereof and 7.5 mg a salt of trospium twice daily, and 50 mg xanomeline and/or the salt thereof and 5 mg a salt of trospium once daily.

In certain embodiments, the administration comprises a dosing schedule of 50 mg xanomeline and/or the salt thereof and 10 mg a salt of trospium twice daily.

In certain embodiments, the administration comprises a dosing schedule of 50 mg xanomeline and/or the salt thereof and 10 mg a salt of trospium thrice daily.

In certain embodiments, the administration comprises a dosing schedule of 50 mg xanomeline and/or the salt thereof and 10 mg a salt of trospium twice daily, and 75 mg xanomeline and/or the salt thereof and 10 mg a salt of trospium once daily.

In certain embodiments, the administration comprises a dosing schedule of 75 mg xanomeline and/or the salt thereof and 10 mg a salt of trospium twice daily, and 50 mg xanomeline and/or the salt thereof and 10 mg a salt of trospium once daily.

In certain embodiments, the low doses are split TID rather than BID. BID keeps the trospium dose as low as possible with a minimum amount of 10 mg of trospium per capsule. In certain embodiments, a starting dose of 50/5 mg or 50/7.5 mg xanomeline/trospium is split TID, so the individual dose 17/2.5 mg xanomeline/trospium.

In one embodiment, an extended-release formulation of trospium chloride is used in combination with xanomeline. In another embodiment, trospium chloride extended-release is administered to a patient from one time to five times during a 24-hour period. In an embodiment, the extended-release of trospium chloride is administered one to three times during a 24-hour period. In another embodiment, from five milligrams to 400 milligrams of trospium chloride, extended-release is used during a 24-hour period. In an embodiment, from 20 milligrams to 200 milligrams of trospium chloride extended-release is used during a 24-hour period.

In one embodiment, 225 mg xanomeline and 40 mg trospium chloride are administered to a patient in a 24-hour period. In another embodiment, 100 mg xanomeline and 20 mg trospium chloride are administered to a patient in a 24-hour period. In another embodiment, 125 mg xanomeline and 20 mg trospium chloride are administered to a patient in a 24-hour period. In another embodiment, 125 mg xanomeline and 30 mg trospium chloride are administered to a patient in a 24-hour period. In another embodiment, 125 mg xanomeline and 40 mg trospium chloride are administered to a patient in a 24-hour period. In another embodiment, 200 mg xanomeline and 40 mg trospium chloride are administered to a patient in a 24-hour period. In another embodiment, 200 mg xanomeline and 80 mg trospium chloride are administered to a patient in a 24-hour period. In another embodiment, 250 mg xanomeline and 60 mg trospium chloride are administered to a patient in a 24-hour period. In another embodiment, 250 mg xanomeline and 80 mg trospium chloride are administered to a patient in a 24-hour period. In another embodiment, 300 mg xanomeline and 40 mg trospium chloride are administered to a patient in a 24-hour period. In another embodiment, 300 mg xanomeline and 60 mg trospium chloride are administered to a patient in a 24-hour period. In another embodiment, 300 mg xanomeline and 80 mg trospium chloride are administered to a patient in a 24-hour period. In another embodiment, 350 mg xanomeline and 40 mg trospium chloride are administered to a patient in a 24-hour period. In another embodiment, 350 mg xanomeline and 60 mg trospium chloride are administered to a patient in a 24-hour period. In another embodiment, 350 mg xanomeline and 80 mg trospium chloride are administered to a patient in a 24-hour period.

In certain embodiments, the total daily dose is between 60 and 200 mg xanomeline and/or a salt thereof and between 6 and 30 mg of a salt of trospium. In certain embodiments, the total daily dose is between 120 and 200 mg xanomeline and/or a salt thereof and between 12 and 20 mg of a salt of trospium. In certain embodiments, the total daily dose is 60 mg xanomeline and/or a salt thereof and of a salt of trospium administered as 20 mg the xanomeline and/or a salt thereof and 2.0 mg the salt of trospium in three doses. In certain embodiments, the total daily dose is 90 mg xanomeline and/or a salt thereof and 9 mg of a salt of trospium administered as 30 mg the xanomeline and/or a salt thereof and 3.0 mg the salt of trospium in three doses. In certain embodiments, the total daily dose is 120 mg xanomeline and/or a salt thereof and 12 mg of a salt of trospium administered as 40 mg the xanomeline and/or a salt thereof and 4.0 mg the salt of trospium in three doses. In certain embodiments, the total daily dose is 150 mg xanomeline and/or a salt thereof and 15 mg of a salt of trospium administered as 50 mg the xanomeline and/or a salt thereof and 5.0 mg the salt of trospium in three doses. In certain embodiments, the total daily dose is 200 mg xanomeline and/or a salt thereof and 20 mg of a salt of trospium administered as 66.7 mg the xanomeline and/or a salt thereof and 6.67 mg the salt of trospium in three doses.

In certain embodiments, reducing the daily dose of the salt of trospium improves the overall tolerability of the xanomeline and/or a salt thereof.

### Randomized Withdrawal and Decreasing Risk of Relapse

The present disclosure provides a method of decreasing the risk of relapse in a patient having dementia-related psychosis, comprising administering a total daily dose of between 25 and 250 mg xanomeline and/or a salt thereof and between 5 and 60 mg of a salt of trospium to the patient. In certain embodiments, when the patient is 55 years or older, the total daily dose is between 25 and 200 mg xanomeline and/or a salt thereof and between 5 and 30 mg of the salt of trospium. In certain embodiments, when the patient is younger than 55 years, the total daily dose is between 100 and 250 mg xanomeline and/or a salt thereof and between 40 and 60 mg of the salt of trospium.

In certain embodiments, patients have psychosis and are treated with xanomeline and trospium with the goal of a clinically meaningful therapeutic benefit, for example, defined as an at least 30% reduction in NPI-C H+D and CGI-I improvement, such as an at least 40% reduction, or an at least 50% reduction. For those patients who improve, they continue on with xanomeline and trospium. In this embodiment, the treatment prevents relapse, providing maintenance therapy to the patient rather than preventing symptom onset; these patients experience an initial recovery using the disclosed method, and then maintain the therapeutic effect with continued use of xanomeline and trospium compared to the same patient taking placebo.

In certain embodiments, relapse is indicated by at least a 40% increase on the Neuropsychiatric Inventory Clinician hallucinations and delusions (NPI-C H+D) score and a Clinical Global Impression-Improvement (CGI-I) score of 6 or 7 at 12 weeks or more of the administration compared to before the administration. In certain embodiments, relapse is indicated by at least a 40% increase on the Neuropsychiatric Inventory Clinician (NPI-C) Core score and a Clinical Global Impression-Improvement (CGI-I) score of 6 or 7 at 12 weeks or more of the administration compared to before the administration. In certain embodiments, relapse is indicated by treatment with an antipsychotic other than the xanomeline and/or a salt thereof and the salt of trospium for dementia-related delusions or hallucinations during the administration. In certain embodiments, relapse is indicated by the patient stopping the administration of the xanomeline and/or a salt thereof and the salt of trospium. In certain embodiments, relapse is indicated by discontinuation of the administration of the xanomeline and/or a salt thereof and the salt of trospium due to lack of efficacy. In certain embodiments, relapse is indicated by the patient being hospitalized for worsening psychosis, agitation, or aggression symptoms during the administration.

In certain embodiments, said administration results in an improvement in the patient's NPI-C Core score. In certain embodiments, said administration results in an improvement in the patient's Cohen-Mansfield Agitation Inventory (CMAI) Total score.

In certain embodiments, said administration results in an improvement in NPI-C Psychosis score, NPI-C Agitation score, NPI-C Aggression score, NPI-C Total score, Clinical Global Impression-Severity (CGI-S) scale score related to psychosis, agitation, and aggression, or a CGI-I scale score related to psychosis, agitation, and aggression. In certain embodiments, the NPI Psychosis score comprises the sum of the hallucination and delusion domain scores.

In certain embodiments, said administration results in an improvement in the NPI-C Core score.

In certain embodiments, prior to the administration, the patient meets clinical criteria for possible or probable Alzheimer's disease.

In certain embodiments, prior to the administration, the patient has a history of psychotic symptoms meeting International Psychogeriatric Association criteria.

In certain embodiments, prior to the administration, the patient has a Clinical Global Impression-Severity scale with a score or at least 4.

In certain embodiments, prior to the administration, the patient has Alzheimer's dementia with moderate to severe delusions, defined as NPI-C Delusion domain score of at least 2 on two of the eight items and a NPI Agitation/Aggression domain score or at least 4.

In certain embodiments, prior to the administration, the patient has a Mini-Mental State Examination score of between 8 and 22.

In certain embodiments, prior to the administration, the patient does not have psychotic symptoms primarily attributable to a condition other than Alzheimer's disease causing dementia.

Each and every method, composition, or use described herein also optionally includes the limitation that the dementia-related psychosis is not psychosis associated with Alzheimer's disease, psychosis associated with Parkinson's disease, psychotic depression, bipolar disorder, bipolar with psychosis.

In some embodiments, the treatment occurs outside of a clinical trial setting.

### Pharmaceutical Compositions

Provided herein is an oral pharmaceutical composition, comprising a plurality of xanomeline beads comprising xanomeline or a salt thereof and optionally a first coating; and a plurality of trospium beads comprising a salt of trospium and optionally a second coating.

In certain embodiments, the salt of trospium is chosen from trospium chloride, trospium bromide, trospium iodide, and trospium saccharinate. In some embodiments, the first coating has the same composition as the second coating.

In certain embodiments, the oral pharmaceutical composition comprises a plurality of xanomeline beads comprising xanomeline or a salt thereof. In certain embodiments, the oral pharmaceutical composition comprises a plurality of trospium beads comprising a salt of trospium.

In certain embodiments, the plurality of xanomeline beads has a core comprising xanomeline or a salt thereof and optionally a first coating. In certain embodiments, the plurality of trospium beads has a core comprising a trospium salt and optionally a second coating.

In certain embodiments, a capsule shell comprising hydroxypropyl methylcellulose (HPMC) containing separate populations of drug beads containing xanomeline tartrate or trospium chloride wherein the drug beads are of comparable size and release the actives rapidly and at substantially similar rates. Following the dissolution of the capsule shell in the stomach, the drug beads may dissolve in the stomach or pass through the pyloric valve into the duodenum intact or partially intact. Still, the two drugs' ratio, both in dissolved form and in undissolved form, remains relatively constant in the gastrointestinal tract until the drugs are absorbed.

The formulation for each drug bead allows substantially similar performance from two actives at different dose ranges. The actives are released into the blood serum at substantially similar rates or achieve a substantially similar Tₘₐₓ. In certain embodiments, a capsule comprises 50 mg xanomeline as the tartrate salt and 10 mg trospium chloride. Fifty mg xanomeline as a free base corresponds to about 76 mg xanomeline tartrate.

A discrepancy in the number of drug beads in the capsule increases the probability that the drug beads' ratio would not remain substantially constant after the beads are released and disperse. Thus, in certain embodiments, the trospium beads are formulated with a lower drug load. Effective doses of trospium and xanomeline are contained in roughly equivalent numbers of beads. Despite the differences in drug loads in certain embodiments, the trospium and xanomeline beads release at roughly similar rates. For example, if the dissolution of the capsules is assessed using a United States Pharmacopeia (USP) dissolution apparatus, the percentage of xanomeline dissolved is substantially equivalent to the percentage of dissolved trospium chloride, such as at 10 min, 20 min, or 30 min.

In certain embodiments, the oral pharmaceutical composition is a capsule comprising a plurality of xanomeline beads, each of said plurality of xanomeline beads having a core comprising between 50 wt. % and 90 wt. % xanomeline tartrate, between 15 wt. % and 65 wt. % microcrystalline cellulose, and between 0 wt. % and 1 wt. % ascorbic acid, and optionally a first coating comprising between 0 wt. % and 2 wt. % talc, wherein each of said plurality of xanomeline beads has a size between 0.6 mm and 0.85 mm; a plurality of trospium beads, each of said plurality of trospium beads having a core comprising between 8 wt. % and 35 wt. % trospium chloride, between 45 wt. % and 50 wt. % microcrystalline cellulose, and between 35 wt. % and 40 wt. % lactose monohydrate, and optionally a second coating comprising between 0 wt. % and 2 wt. % talc, wherein each of said plurality of trospium beads has a size between 0.6 mm and 0.85 mm; and the plurality of xanomeline beads and the plurality of trospium beads each having a dissolution rate of more than about 95% within about the first 20 minutes following entry of the capsule into an aqueous solution.

In certain embodiments, each of the xanomeline beads comprises 66 wt. % xanomeline tartrate, 33 wt. % microcrystalline cellulose, 0.5 wt. % ascorbic acid, and 0.5 wt. % talc.

In certain embodiments, the plurality of trospium beads have a core comprising between 4 wt. % and 7 wt. % trospium chloride, between 45 wt. % and 60 wt. % microcrystalline cellulose, and between 35 wt. % and 50 wt. % lactose monohydrate, and optionally a second coating comprising between 0 wt. % and 2 wt. % talc.

In certain embodiments, each of the trospium beads comprises 17.7 wt. % trospium chloride, 46.8 wt. % microcrystalline cellulose, 35 wt. % lactose monohydrate, and 0.5 wt. % talc.

In certain embodiments, each of the trospium beads comprises 4.4 wt. % trospium chloride, 54.5 wt. % microcrystalline cellulose, 40.6 wt. % lactose monohydrate, and 0.5 wt. % talc. In certain embodiments, each of the trospium beads comprises a core comprising 4.4 wt. % trospium chloride, 54.5 wt. % microcrystalline cellulose, and 40.6 wt. % lactose monohydrate, and optionally a coating comprising 0.5 wt. % talc.

In certain embodiments, the capsule comprises an oral pharmaceutical composition wherein each of said plurality of xanomeline beads having a core comprising between 66 wt. % xanomeline tartrate, between 33 wt. % microcrystalline cellulose, 0.5 wt. % ascorbic acid, and a coating comprising 0.5 wt. % talc; each of said a plurality of trospium beads having a core comprising between 17.7 wt. % trospium chloride, 46.8 wt. % microcrystalline cellulose, 35 wt. % lactose monohydrate, and a coating comprising 0.5 wt. % talc.

The medicament may also include one or more pharmaceutically acceptable salts. The medicament may include one or more pharmaceutically-acceptable carriers. The medicament may be administered orally. The medicament may be delivered orally using tablets, troches, liquids, emulsions, suspensions, drops, capsules, caplets or gel caps, and other methods of oral administration known to one skilled in the art.

The medicament may be in a dosage form that immediately releases the drug. In an alternative embodiment, the medicament may have a controlled release dosage form.

The medicament may be in dosage forms that use other controlled-release formulations known to one in the art.

In another embodiment, the medicament is combined with one or more therapies, including psychotherapy and drugs. Therapeutic agents include, but are not limited, to antipsychotics, anxiolytics, anti-depressants, sedatives, tranquilizers, analgesics, and other pharmacological interventions known to one skilled in the art. A therapeutic agent may fall under the category of more than one drug. For instance, benzodiazepines can be considered anxiolytics, sedatives, and tranquilizers.

### Bead/Core Excipients

The bead or core can comprise one or more excipients. In one embodiment, the excipients include one or more fillers, binders, and surfactants. Other optional ingredients include, but are not limited to, glidants, lubricants, disintegrants, swelling agents, and antioxidants. The xanomeline or a pharmaceutically acceptable salt thereof and the salt of trospium may be in separate matrices within the same medicament.

The amount of xanomeline free base in the core can be at least 10 wt. % or at least 15 wt. %, or at least 20 wt. %, or at least 25 wt. %, or at least 30 wt. %. For example, the amount of xanomeline tartrate can be at least 50 wt. %, or at least 55 wt. %, or at least 60 wt. %, or at least 65 wt. %, or at least 70 wt. %, or at least 75 wt. %, or at least 80 wt. %, or at least 85 wt. % of the core, in a range of about 60 wt. % to about 90 wt. % or about 65 wt. % to about 85 wt. %. It is understood that all ranges including these values as endpoints are contemplated, for example, at least between about 15 wt. % and about 90 wt. %, between about 20 wt. % and about 85 wt. %, between about 30 wt. % and about 85 wt. %, or between about 50 wt. % and about 90 wt. %. In certain embodiments, the xanomeline beads comprise between 30 wt. % and 80 wt. % xanomeline tartrate, such as 66 wt. % xanomeline tartrate.

The amount of trospium salt in the core can be at least 10 wt. % or at least 15 wt. %, or at least 20 wt. %, or at least 25 wt. %, or at least 30 wt. %. For example, the amount of trospium chloride can be at least 50 wt. %, or at least 55 wt. %, or at least 60 wt. %, or at least 65 wt. %, or at least 70 wt. %, or at least 75 wt. %, or at least 80 wt. %, or at least 85 wt. % of the core, in a range of about 60 wt. % to about 90 wt. % or about 65 wt. % to about 85 wt. %. It is understood that all ranges including these values as endpoints are contemplated, for example, at least between about 15 wt. % and about 90 wt. %, between about 20 wt. % and about 85 wt. %, between about 30 wt. % and about 85 wt. %, or between about 50 wt. % and about 90 wt. %. In certain embodiments, the trospium is trospium chloride. In certain embodiments, the trospium beads comprise between 8 wt. % and 35 wt. % trospium chloride, such as 17.7 wt. % trospium chloride.

In a further embodiment, the matrix comprises a polymer, for example, to modify the release profile of the active in the matrix. In a further embodiment, the polymer comprises a water-soluble polymer. In a further embodiment, the water-soluble polymer is selected from Eudragit™ RL, polyvinyl alcohol, polyvinylpyrrolidone, methylcellulose, hydroxypropyl cellulose, hydroxypropylmethylcellulose, polyethylene glycol, and mixtures thereof. In a further embodiment, the polymer comprises a water-insoluble polymer. In a further embodiment, the water-insoluble polymer is selected from Eudragit™ RS, ethylcellulose, cellulose acetate, cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(propylene), poly(ethylene terephthalate), poly(vinyl isobutyl ether), poly(vinyl acetate), poly(vinyl chloride), polyurethane, and mixtures thereof.

Fillers include, but are not limited to, lactose, saccharose, glucose, starch, microcrystalline cellulose, microfine cellulose, mannitol, sorbitol, calcium hydrogen phosphate, aluminum silicate, amorphous silica, and sodium chloride, starch, and dibasic calcium phosphate dihydrate. In one embodiment, the filler is not water-soluble, although it may absorb water. In one embodiment, the filler is a spheronization aid. Spheronization aids can include one or more of crospovidone, carrageenan, chitosan, pectinic acid, glycerides, β-cyclodextrin (β-CD), cellulose derivatives, microcrystalline cellulose, powdered cellulose, polyplasdone crospovidone, and polyethylene oxide. In one embodiment, the filler includes microcrystalline cellulose.

The amount of filler in the xanomeline core is not particularly limited. In embodiments, the amount of filler (e.g., microcrystalline cellulose) can range from about 10 wt. % to about 70 wt. %, or about 16 wt. % to about 23 wt. %, or at least 19 wt. % or at least 19.5 wt. %, for example, about 20 wt. %. In certain embodiments, the xanomeline beads comprise between about 15 wt. % and about 65 wt. % microcrystalline cellulose, such as between about 15 wt. % and about 20 wt. %, between about 20 wt. % and about 25 wt. %, between about 25 wt. % and about 30 wt. %, between about 30 wt. % and about 35 wt. %, between about 35 wt. % and about 40 wt. %, between about 40 wt. % and about 45 wt. %, between about 45 wt. % and about 50 wt. %, between about 50 wt. % and about 55 wt. %, between about 55 wt. % and about 60 wt. %, or between about 60 wt. % and about 65 wt. %. In certain embodiments, the xanomeline beads comprise 33.5 wt. % microcrystalline cellulose.

The amount of filler in the trospium core is not particularly limited. In embodiments, the amount of filler (e.g., microcrystalline cellulose or lactose) can be in a range of about 10 wt. % to about 80 wt. %, or about 16 wt. % to about 23 wt. %, or at least 19 wt. % or at least 19.5 wt. %, for example about 20 wt. %. In certain embodiments, the trospium beads comprise between 25 wt. % and 80 wt. % microcrystalline cellulose, such as between about 25 wt. % and 30 wt. %, between about 30 wt. % and 35 wt. %, between about 35 wt. % and 40 wt. %, between about 40 wt. % and 45 wt. %, between about 45 wt. % and 50 wt. %, between about 50 wt. % and 55 wt. %, between about 55 wt. % and 60 wt. %, between about 60 wt. % and 65 wt. %, between about 65 wt. % and 70 wt. %, between about 70 wt. % and 75 wt. %, or between about 75 wt. % and 80 wt. %. In certain embodiments, the trospium beads comprise 46.8 wt. % microcrystalline cellulose.

In certain embodiments, the trospium beads comprise between 15 wt. % and 70 wt. % lactose monohydrate, such as between about 15 wt. % and 20 wt. %, between about 20 wt. % and 25 wt. %, between about 25 wt. % and 30 wt. %, between about 30 wt. % and 35 wt. %, between about 35 wt. % and 40 wt. %, between about 40 wt. % and 45 wt. %, between about 45 wt. % and 50 wt. %, between about 50 wt. % and 55 wt. %, between about 55 wt. % and 60 wt. %, between about 60 wt. % and 65 wt. %, or between about 65 wt. % and 70 wt. %. In certain embodiments, the trospium beads comprise 35 wt. % lactose monohydrate.

Binders include, but are not limited to, cellulose ethers, methylcellulose, ethylcellulose, hydroxyethylcellulose, propyl cellulose, hydroxypropyl cellulose, lower-substituted hydroxypropyl cellulose, hydroxypropylmethylcellulose (hypromellose, e.g., hypromellose 2910, Methocel™ E), carboxymethyl cellulose, starch, pregelatinized starch, acacia, tragacanth, gelatin, polyvinyl pyrrolidone (povidone), cross-linked polyvinyl pyrrolidone, sodium alginate, microcrystalline cellulose, and lower-alkyl-substituted hydroxypropyl cellulose. In one embodiment, the binders are selected from wet binders. In one embodiment, the binder is selected from cellulose ethers, e.g., hypromellose.

The amount of binder in the xanomeline core is not particularly limited. In embodiments, the amount of binder (e.g., hypromellose) can be between about 1 wt. % and about 10 wt. %, between about 2 wt. % and about 8 wt. %, or between about 4 wt. % and about 6 wt. %, for example, about 5 wt. %.

The amount of binder in the trospium core is not particularly limited. In embodiments, the amount of binder (e.g., hypromellose) can be between about 1 wt. % and about 10 wt. %, between about 2 wt. % and about 8 wt. %, or between about 4 wt. % and about 6 wt. %, for example, about 5 wt. %.

Surfactants include, but are not limited to, anionic surfactants, including sodium lauryl sulfate, sodium deoxycholate, dioctyl sodium sulfosuccinate, and sodium stearyl fumarate, nonionic surfactants, including polyoxyethylene ethers and polysorbate 80, and cationic surfactants, including quaternary ammonium compounds. In one embodiment, the surfactant is selected from anionic surfactants, e.g., sodium lauryl sulfate.

The amount of surfactant, e.g., as a processing aid, is not particularly limited in the xanomeline core. In embodiments, the amount of surfactant (e.g., microcrystalline cellulose) can range between about 0.1 wt. % and about 1 wt. %, between about 0.2 wt. % and about 0.8 wt. %, or between about 0.4 wt. % and about 0.6 wt. %, for example, about 0.5 wt. %.

The amount of surfactant, e.g., as a processing aid, is not particularly limited in the trospium core. In embodiments, the amount of surfactant (e.g., sodium lauryl sulfate) can range between about 0.1 wt. % and about 1 wt. %, between about 0.2 wt. % and about 0.8 wt. %, or between about 0.4 wt. % and about 0.6 wt. %, for example, about 0.5 wt. %.

Disintegrants include, but are not limited to, starch, sodium cross-linked carboxymethyl cellulose, carmellose sodium, carmellose calcium, cross-linked polyvinyl pyrrolidone, and sodium starch glycolate, low-substituted hydroxypropyl cellulose, and hydroxypropyl starch.

Glidants include but are not limited to polyethylene glycols of various molecular weights, magnesium stearate, calcium stearate, calcium silicate, fumed silicon dioxide, magnesium carbonate, magnesium lauryl sulfate, aluminum stearate, stearic acid, palmitic acid, cetanol, stearol, and talc.

Lubricants include, but are not limited to, stearic acid, magnesium stearate, calcium stearate, aluminum stearate, and siliconized talc. In certain embodiments, the xanomeline beads comprise between 0 wt. % and 2 wt. % talc, such as 0.5 wt. % talc. In certain embodiments, the trospium beads comprise between 0 wt. % and 2 wt. % talc, such as 0.5 wt. % talc.

In certain embodiments, the formulation further comprises one or more antioxidants. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. In certain embodiments, the formulation comprises less than 1 wt. % antioxidant, such as 0.9 wt. %, 0.8 wt. %, 0.7 wt. %, 0.6 wt. %, 0.5 wt. %, 0.4 wt. %, 0.3 wt. %, 0.2 wt. %, 0.1 wt. %, 0.09 wt. %, 0.08 wt. %, 0.07 wt. %, 0.06 wt. %, 0.05 wt. %, 0.04 wt. %, 0.03 wt. %, 0.02 wt. %, or 0.01 wt. %.

In certain embodiments, the oral pharmaceutical composition further comprises ascorbic acid. In certain embodiments, the oral pharmaceutical composition comprises between 0.2 wt. % and 1 wt. % ascorbic acid. In certain embodiments, the oral pharmaceutical composition comprises about 0.5 wt. % ascorbic acid. In certain embodiments, the oral pharmaceutical composition further comprises butylated hydroxytoluene. In certain embodiments, the oral pharmaceutical composition comprises between 0.01 wt. % and 0.1 wt. % butylated hydroxytoluene. In certain embodiments, the oral pharmaceutical composition comprises about 0.05 wt. % butylated hydroxytoluene. In certain embodiments, the formulation comprises about 0.05 wt. % BHT or 0.5 wt. % ascorbic acid. In certain embodiments, the antioxidant is present in the xanomeline core or the xanomeline beads.

In certain embodiments, the xanomeline beads comprise between 30 wt. % and 80 wt. % xanomeline tartrate, between 15 wt. % and 65 wt. % microcrystalline cellulose, and between 0 wt. % and 2 wt. % talc. In certain embodiments, the trospium beads comprise between 0.2 wt. % and 2 wt. % talc, such as 0.5 wt. % talc. In certain embodiments, the trospium beads comprise between 8 wt. % and 35 wt. % trospium chloride, between 25 wt. % and 80 wt. % microcrystalline cellulose, between 15 wt. % and 70 wt. % lactose monohydrate, and between 0.2 wt. % and 2 wt. % talc.

In certain embodiments, the xanomeline tartrate drug beads comprise 66 wt. % xanomeline tartrate, 33-34 wt. % microcrystalline cellulose, and 0.5 wt. % talc. In certain embodiments, the trospium chloride beads comprise 17.7 wt. % trospium chloride, 46.8 wt. % microcrystalline cellulose, 35 wt. % lactose monohydrate, and 0.5 wt. % talc. In this example, the xanomeline tartrate beads contain at least about 2.5 times as much xanomeline as the trospium chloride beads contain trospium chloride.

Depending on dosing requirements, capsules can be prepared with different amounts of xanomeline tartrate and trospium chloride beads. In various embodiments, capsules contain 25 mg xanomeline and 10 mg trospium chloride, 50 mg xanomeline and 10 mg trospium chloride, 50 mg xanomeline and 20 mg trospium chloride, 75 mg xanomeline and 10 mg trospium chloride, 75 mg xanomeline and 20 mg trospium chloride, 125 mg xanomeline and 30 mg trospium chloride, or 125 mg xanomeline and 40 mg trospium chloride. In certain embodiments, the capsule contains 25 mg xanomeline as xanomeline tartrate and 10 mg trospium chloride. In certain embodiments, the capsule contains 50 mg xanomeline as xanomeline tartrate and 10 mg trospium chloride. In certain embodiments, the capsule contains 50 mg xanomeline as xanomeline tartrate and 20 mg trospium chloride. In certain embodiments, the capsule contains 75 mg xanomeline as xanomeline tartrate and 10 mg trospium chloride. In certain embodiments, the capsule contains 75 mg xanomeline as xanomeline tartrate and 20 mg trospium chloride. In certain embodiments, the capsule contains 125 mg xanomeline as xanomeline tartrate and 20 mg trospium chloride. In certain embodiments, the capsule contains 125 mg xanomeline as xanomeline tartrate and 40 mg trospium chloride. In certain embodiments, the capsule contains 150 mg xanomeline and 20 mg trospium chloride. In certain embodiments, the capsule contains 150 mg xanomeline and 30 mg trospium chloride. In certain embodiments, the capsule contains 150 mg xanomeline and 40 mg trospium chloride. In certain embodiments, the capsule contains 175 mg xanomeline and 20 mg trospium chloride. In certain embodiments, the capsule contains 175 mg xanomeline and 30 mg trospium chloride. In certain embodiments, the capsule contains 175 mg xanomeline and 40 mg trospium chloride.

In another embodiment, the medicament contains from five milligrams to 700 milligrams of xanomeline. In an embodiment, the medicament contains from 25 milligrams to 300 milligrams of xanomeline.

In another embodiment, the medicament contains from one milligram to 400 milligrams of trospium chloride. In an embodiment, the medicament contains from 6.5 milligrams to 200 milligrams of trospium chloride.

In one embodiment, trospium chloride extended-release is used as the trospium chloride in the medicament. In another embodiment, the medicament contains from one milligram to 400 milligrams of trospium chloride extended-release. In an embodiment, the medicament contains from 6.5 milligrams to 200 milligrams of trospium chloride extended-release.

In an embodiment, the medicament contains 75 mg or 225 milligrams of xanomeline, and the same medicament contains 20 mg or 40 milligrams of trospium chloride. In another embodiment, the medicament contains 75 mg or 225 milligrams of xanomeline, and a different medicament to be co-administered contains 20 mg or 40 milligrams of trospium chloride.

It has been observed that, in this patient population aged 55 years and older, that a dose of <40 mg trospium chloride performed better than did higher doses. In certain embodiments, the dose of trospium chloride is <30 mg, such as <20 mg. The trospium doses are co-administered to patients in need thereof with at least 200 mg xanomeline. This relationship can also be expressed as ratios between xanomeline and trospium, where a ratio of 5:1 xanomeline/trospium (as used in the EMERGENT-1 trial in patients with schizophrenia), led to undesirable tolerability in the elderly, where was ratios of 6:1 xanomeline/trospium or 7.5:1 xanomeline/trospium were better tolerated. In certain embodiments, the ratio of xanomeline to trospium is greater than 5:1, such as greater than 6:1, greater than 7.5:1, or greater than 10:1. In certain embodiments, the ratio of xanomeline to trospium is 10:1.

### Bead Coatings

In other embodiments, the beads may be coated with functional or non-functional coatings, such as aesthetic, handling, or stability. In certain embodiments, the beads might be coated with a pH-sensitive coating so that they do not dissolve in the low pH of the stomach. A nonfunctional coating might be used to maintain chemical separation between the beads or for cosmetic reasons.

In certain embodiments, the beads comprise a coating of talc, which, for example, aids flowability and mixing. Generally, the beads comprise a coating of between 0 wt. % and 2 wt. % talc, such as 0 wt. % to 0.5 wt. % talc, 0.4 wt. % to 0.6 wt. % talc, 0.5 wt. % to 1 wt. % talc, 1 wt. % to 1.5 wt. % talc, or 1.5 wt. % to 2 wt. % talc. In certain embodiments, the beads comprise a coating of 0.5 wt. % talc (in relation to the total weight of the bead). In certain embodiments, the first coating comprises talc. In certain embodiments, the second coating comprises talc. In a further embodiment, the controlled release formulation comprises a semi-permeable coating. The xanomeline and trospium chloride may be in different coatings in the same formulation. The xanomeline and trospium chloride can be in different coatings in different formulations or dosing vehicles in another embodiment. In a further embodiment, the semi-permeable coating comprises a polymer. In a further embodiment, the controlled release formulation comprises a matrix that suspends the xanomeline and trospium chloride.

In certain embodiments, the distribution of coating thicknesses can be stated in the weight gain of coating material based on the total weight of the coated beads. Thus, in one embodiment, the distribution of coating thicknesses is at least 2% based on the total weight of the coated beads. In another embodiment, the distribution of coating thicknesses is at least 3%. In another embodiment, the distribution of coating thicknesses is at least 4%. In another embodiment, the distribution of coating thicknesses is at least 5%. In another embodiment, the distribution of coating thicknesses is at least 6%. In another embodiment, the distribution of coating thicknesses is at least 7%. In another embodiment, the distribution of coating thicknesses is at least 8%. In another embodiment, the distribution of coating thicknesses is at least 9%. In another embodiment, the distribution of coating thicknesses is at least 10%. In another embodiment, the distribution of coating thicknesses is at least 11%. In another embodiment, the distribution of coating thicknesses is at least 12%. In another embodiment, the distribution of coating thicknesses is at least 13%. In another embodiment, the distribution of coating thicknesses is at least 14%.

For example, the difference in coating thickness from bead to bead can be in a range of +/−1-7% based on the coated beads' total weight. The distribution of coating thicknesses can be between about 2% and about 14% based on the weight of the coated beads, such as between about 3% and about 13%, between about 4% and about 12%, between about 5% and about 11%, between about 6% to about 10%, between about 7% and 9%, between about 3% and 14%, between about 4% and 14%, between about 4% and 13%, or between 4% and about 12%.

In one embodiment, the absorption (area under the curve, AUC) of the dosage form when dosed orally is advantageously increased, compared to other dosage forms of xanomeline or trospium chloride. Without intending to be bound by any theory, the increase in absorption is influenced by the dosage form exhibiting a pseudo-extended release profile. The pseudo-extended release profile is influenced by one or more factors, including distribution of coating thicknesses when present, distribution of bead particle sizes, and the beads having irregular bead shapes. For example, in an embodiment wherein the beads have a distribution of coating thicknesses, for beads with a relatively thin coating, the coating completely dissolves at the trigger pH relatively quickly to release the xanomeline and/or trospium chloride compositions, whereas for beads having a relatively thick coating the coating takes somewhat longer to completely dissolve and release the xanomeline and/or trospium chloride compositions. In an embodiment where the beads have a distribution of particle sizes and/or irregular bead shapes, the gut transit time of the beads could be varied due to bead size and/or shape, such that the transit time until reaching the coating dissolution pH is varied, thus contributing to a pseudo-extended release profile. In another embodiment, the dosage form exhibits substantially equivalent (e.g., bioequivalent) Cₘₐₓ and/or AUC characteristics when administered orally inside a capsule shell or without a capsule shell.

In certain embodiments, the dosage form provides a progressive and predictable absorption curve. In one embodiment, the Tₘₐₓ of the dosage form, when dosed orally, is more stable on a dose-to-dose basis because the beads are individually coated. A predictable, consistent Tₘₐₓ is advantageous for accomplishing a more consistent, sustained therapeutic effect. For example, process-related variations in coating thickness or other influences on coating dissolution affect only a fraction of the xanomeline and trospium chloride in the dosage form. They tend to lead to pseudo-extended release behavior. In contrast, coated capsules comprising xanomeline and trospium chloride microspheres exhibit significant variability in absorption time from the capsule to capsule.

In certain embodiments, the oral pharmaceutical composition comprises xanomeline and/or a salt thereof and trospium chloride for treating a muscarinic disorder in a patient in need thereof, which when administered to the patient in need thereof, the composition is sufficient to provide an in-vivo plasma profile comprising a median Tₘₐₓ for xanomeline of 2 hours and a median Tₘₐₓ for trospium of 1 hour. In certain embodiments, the in-vivo plasma profile further comprises a mean dose-normalized Cₘₐₓ of between 48.5 and 121.3 pg/mL/mg. In certain embodiments, the in-vivo plasma profile further comprises a mean dose-normalized Cₘₐₓ of trospium of between 156 and 375 pg/mL/mg. In certain embodiments, the in-vivo plasma profile further comprises a mean dose-normalized AUC₀₋₁₂ of xanomeline of between 263 and 577 hr·pg/mL/mg. In certain embodiments, the in-vivo plasma profile further comprises a mean dose-normalized AUC₀₋₁₂ of trospium of between 881 and 2024 hr·pg/mL/mg. In certain embodiments, the in-vivo plasma profile further comprises a mean Cₘₐₓ of trospium at 7850±3360 pg/mL. In certain embodiments, the in-vivo plasma profile further comprises a mean AUC₀₋₁₂ of 41900±15500 hr·pg/mL.

In another embodiment, the dosage form exhibits advantageous storage stability, e.g., measured by the amount of xanomeline present following storage and/or by the total amount of related substances. The storage stability can be assessed following storage at typical ambient conditions (e.g., 25° C. and 60% relative humidity) or accelerated stability conditions involving increased temperature and/or humidity.

The dosage form and methods are contemplated to include embodiments of any combination of one or more of the additional optional elements, features, and steps further described below (including those shown in the figures and Examples) unless stated otherwise. Reference to a bead and properties thereof apply equally to a collection of beads (e.g., a plurality of such beads). Likewise, referring to a core and properties thereof apply equally to a collection of cores (e.g., a plurality of such cores).

The enteric (gastro-resistant) coating material, e.g., polymer, can be one that will dissolve in intestinal juices at a pH level higher than that of the stomach, e.g., a pH of greater than 4.5, such as within the small intestine, and therefore permit the release of the active substance in the regions of the small intestine and substantially not in the upper portion of the GI tract. In one embodiment, the enteric material begins to dissolve in an aqueous solution at pH between about 4.5 and about 5.5. In another embodiment, the enteric material rapidly dissolves in an aqueous solution at a pH of about 5. In another embodiment, the enteric material rapidly dissolves in an aqueous solution at a pH of about 5.5.

For example, pH-sensitive materials do not significantly dissolve until the dosage form has emptied from the stomach. The small intestine's pH gradually increases from about 4.5 to about 6.5 in the duodenal bulb to about 7.2 in the small intestine's distal portions (ileum). To provide predictable dissolution corresponding to the small intestine transit time of about 3 hours (e.g., 2-3 hours) and permit reproducible release therein, the coating should begin to dissolve within the pH range of the duodenum and continue to dissolve at the pH range within the small intestine. Therefore, the amount (thickness) of enteric coating should be substantially dissolved during the about three-hour transit time within the small intestine (e.g., the proximal and mid-small intestine).

Suitable enteric (gastro-resistant) materials include, but are not limited to, cross-linked polyvinyl pyrrolidone; non-crosslinked polyvinylpyrrolidone; hydroxypropylmethyl cellulose phthalate, hydroxypropylmethyl cellulose acetate succinate, cellulose acetate succinate; cellulose acetate phthalate, hydroxypropylmethyl cellulose acetate succinate, cellulose acetate trimellitate; starch acetate phthalate; polyvinyl acetate phthalate; carboxymethyl cellulose; methyl cellulose phthalate; methyl cellulose succinate; methyl cellulose phthalate succinate; methyl cellulose phthalic acid half ester; ethyl cellulose succinate; carboxymethylamide; potassium methacrylate divinylbenzene copolymer; polyvinyl alcohols; polyoxyethylene glycols; polyethylene glycol; sodium alginate; galactomannan; carboxypolymethylene; sodium carboxymethyl starch; copolymers of acrylic acid and/or methacrylic acid with a monomer selected from the following: methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate, or octadecyl acrylate, e.g. Eudragit™-L and -S series, including L 100-55, L 30 D-55, L 100, S 100, L 12.5, and S 12.5, available from Evonik Industries; polyvinyl acetate; fats; oils; waxes; fatty alcohols; shellac; zein; gluten; ethylacrylate-maleic acid anhydride copolymer; maleic acid anhydride-vinyl methyl ether copolymer; styrol-maleic acid copolymer; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); and polyurethane. A combination of enteric materials may also be used. In one embodiment, the enteric material rapidly dissolves at pH 5.5 and higher to provide fast dissolution in the upper bowel. For example, the enteric material can be selected from a copolymer of methacrylic acid and methyl methacrylate and a copolymer of methacrylic acid and ethyl acrylate. For example, an enteric polymer is poly(methacrylic acid co-ethyl acrylate) 1:1 (Eudragit™ L 30 D-55 and Eudragit™ L 100-55).

Other suitable examples of enteric coating coatings include beeswax and glyceryl monostearate; beeswax, shellac and cellulose; and cetyl alcohol, mastic and shellac, and shellac and stearic acid; polyvinyl acetate and ethyl cellulose; and a neutral copolymer of polymethacrylic acid esters (Eudragit™ L 30D); copolymers of methacrylic acid and methacrylic acid methylester, or a neutral copolymer of polymethacrylic acid esters containing metallic stearates. Such coatings comprise mixtures of fats and fatty acids, shellac and shellac derivatives, and cellulose acid phthalates, e.g., those with free carboxyl content.

One or more plasticizers can be added to enteric polymers to increase their pliability and reduce brittleness, as known in the art. Suitable plasticizers include, for example, butyl citrates, triethyl citrate, diethyl phthalate, dibutyl sebacate, polyethylene glycols (PEGs, such as PEG 6000), acetyl triethyl citrate, and triacetin. In one embodiment, the plasticizer is triethyl citrate. While some enteric materials are flexible and do not require plasticizers, more brittle polymers (e.g., Eudragit™ L/S types, Eudragit™ RL/RS, and Eudragit™ FS 30 D) benefit from plasticizers, for example, ranging from between 5 wt. % and 30 wt. % based on the dry polymer mass, between about 8 wt. % and about 12 wt. % triethyl citrate with poly(methacrylic acid co-ethyl acrylate) 1:1.

In certain embodiments, the enteric coatings comprise one or more anti-tacking agents (antiadherents) to reduce the film's tackiness and prevent agglomeration, as it is known in the art. Suitable anti-tacking agents include, but are not limited to, talc, glyceryl monostearate, fumed silica (e.g., Aerosil™ 200), precipitated silica (e.g., Sipernat™ PQ), and magnesium stearate. Anti-tacking agents can be used in any suitable quantity, for example ranging between about 10 wt. % and 100 wt. % based on dry polymer mass, between about 10 wt. % and about 50 wt. %, between about 10 wt. % and about 30 wt. %, or between about 15 wt. % and about 30 wt. %. For example, in one embodiment, it ranges between 15 wt. % and about 30 wt. % based on dry polymer mass.

One or more surfactants can also be added to an enteric coating mixture to increase substrate wettability and/or stabilize suspensions, as it is known in the art. Surfactants include Polysorbate 80, sorbitan monooleate, sodium dodecyl sulfate, and other surfactants described herein.

Any suitable process can form the enteric coating. Coating processes include pan coating, fluid bed coating, and dry coating (e.g., heat dry coating and electrostatic dry coating). Pan coating and fluid bed coating using solvent are well-established processes. In liquid coating, the enteric material and optional excipients (e.g., pigments, plasticizers, anti-tacking agents) are mixed in an organic solvent or water to form a solution or dispersion. The coating solution or dispersion is sprayed into solid dosage forms in a pan coater or a fluid bed dryer and dried by hot air. For example, in a Wurster fluid bed coating process, the coating fluid is sprayed from the fluid bed apparatus's bottom. Alternatively, the coating fluid is applied by top spraying. In certain embodiments, a tangential spray is applied.

The amount of enteric material applied is sufficient to achieve the desired acid resistance and release characteristics. For example, in one embodiment, the amount of enteric coating meets USP <711> requirements (USP 36-NF 31) for delayed-release dosage forms, thereby not releasing 10.0 wt. % of the drug after 2 hours in 0.1 N HCl. In certain embodiments, the formulation releases at least 80% of the active in 20 minutes in pH 6.8 buffer solution, e.g., using a dissolution method of USP 36-NF 31 section <711>.

In one embodiment, the enteric coating is present in an amount in a range between about 10% and 40%, or between 25% and about 35% as measured by the weight gain compared to the uncoated particle cores, or ranging between about 25% and about 31% weight gain, between about 27% and about 31% weight gain, or between about 28.5% and about 31% weight gain, based on the weight of the uncoated particle cores.

The formulation can include a capsule shell in which the beads are disposed. Soft and hard capsule shells are known. The capsule shell is a hard-capsule shell in one embodiment, e.g., a gelatin capsule shell or a vegetable-based hard capsule shell. In certain embodiments, the capsule shell comprises one or more enteric coatings described herein. During accelerated storage, gelatin capsules may collapse. Thus, in certain embodiments, the formulation can include a hydroxypropyl methylcellulose capsule shell.

Thus, for example, one embodiment combining various of the features described above includes a pharmaceutical dosage form comprising a plurality of xanomeline beads, the beads comprising a core comprising xanomeline tartrate, a filler (optionally microcrystalline cellulose), a binder (optionally hypromellose), and an enteric coating (optionally Eudragit™ L 30 D-55) surrounding the core, wherein the plurality of beads has a distribution of particle sizes ranging between about 0.7 mm and about 2.5 mm, wherein the enteric coating ranges between about 20% and about 40% based on the weight of the bead cores, and wherein the beads are disposed in a capsule shell.

### Bead Size and Shape

The plurality of beads has a distribution of particle sizes. The plurality of beads has bead shapes. The plurality of beads has a distribution of coating thicknesses when present.

Beads having a distribution of particle sizes were shown to exhibit advantageous pharmacokinetics. Without intending to be bound by any theory, it is contemplated that the pharmacokinetics are influenced by the plurality of beads having a distribution of core sizes.

In one embodiment, the particle sizes of the beads range between about 0.4 mm and about 1.2 mm, such as between about 0.4 mm and about 0.5 mm, between about 0.5 mm and about 0.6 mm, between about 0.6 mm and about 0.7 mm, between about 0.7 mm and about 0.8 mm, between about 0.8 mm and about 0.9 mm, between about 0.9 mm and about 1.0 mm, between about 1.0 mm and about 1.1 mm, or between about 1.1 mm and about 1.2 mm. In certain embodiments, the size of the xanomeline beads is between about 0.425 mm and about 1.18 mm. In certain embodiments, the size of the xanomeline beads is between about 0.6 mm and about 0.85 mm. In certain embodiments, the size of the trospium beads is between about 0.425 mm and about 1.18 mm. In certain embodiments, the size of the trospium beads is between about 0.6 mm and about 0.85 mm.

The beads or bead mixtures may be used, for example, in suspensions, filled into capsules, compressed into tablets, or filled into sachets. One or more types of modified release beads can be mixed and encapsulated or used as a sprinkle on the subject's food. In certain embodiments, the oral solid dosage form may be any of these forms. In certain embodiments, the dosage form is a capsule.

As the particle size of the beads becomes too small, the variability in the content of the active increases. As the particle size becomes too large, the beads are too large for drug products labeled to be administered via sprinkling (e.g., on applesauce or other soft foods, such as jellies) and swallowed without chewing or administered via an enteral feeding tube. Also, as the particle size increases, the larger particles get coated more than the smaller particles, resulting in lower relative assay than smaller particles. Relatively more beads are needed to meet the label strength per capsule. Filling a capsule shell with enough large particles to meet the label strength per capsule becomes difficult or impossible (e.g., to fill a size 0 capsule to a 75-mg strength of xanomeline-free base).

In one embodiment, the beads are formulated into capsules, e.g., with an encapsulation machine. Various capsule sizes may accommodate the strength and fill weight of the target formulations. Capsule size ranges from 00 to 5 for fill weights ranging between about 15 mg and about 630 mg.

The beads can be sorted (e.g., via sieving) to the desired particle size. In certain embodiments, the particle size range is any particle size range or combination described above regarding the cores. In one embodiment, the particle size range is the same as the particle size range of the uncoated cores. For example, the beads can be sieved such that 5% or less of the bead cores by weight is retained on a #12 mesh (1.68 mm) screen, and 10% or less by weight pass through a #20 mesh (0.84 mm) screen.

### Method of Making

Provided is a method for preparing an oral pharmaceutical composition comprising admixing beads comprising a plurality of xanomeline beads comprising xanomeline or a pharmaceutically acceptable salt thereof with a plurality of trospium beads comprising a salt of trospium, such as trospium chloride. In certain embodiments, the method further comprises formulating the admixed beads into capsules.

Also disclosed herein are a method for preparing the dosage form, comprising coating a core comprising xanomeline or a pharmaceutically acceptable salt thereof and an excipient with an enteric polymer to form the enteric coating, and coating a core comprising trospium chloride or a pharmaceutically acceptable salt thereof and an excipient with an enteric polymer to form the enteric coating. Optionally, the core can be formed by a wet granulation method. Optionally, drug beads are sorted (e.g., via sieving) to a desired particle size range before enteric coating and optionally again following enteric coating.

The drug beads may be made by different processes, including, but not limited to, spheronizing an extruded wet mass and coating of inert core spheres in a fluidized bed. In certain embodiments, the beads are prepared by extrusion and spheronization.

The beads are formulated to flow freely and to be compatible with modern encapsulation equipment. In some embodiments, the beads are blended to form a uniform mixture filled into capsules in a single stage. In other embodiments, the beads are filled separately into capsules using a two-stage capsule filler.

Any suitable process can form the cores comprising xanomeline or pharmaceutically acceptable salts thereof. In one embodiment, the core is formed by granulating a mixture of xanomeline or a pharmaceutically acceptable salt thereof with an excipient and milling to a desired particle size range. In another embodiment, the core can be formed by extrusion and spheronization of a mixture of xanomeline or a pharmaceutically acceptable salt thereof with an excipient.

Any suitable process can form the cores comprising trospium chloride or pharmaceutically acceptable salts thereof. In one embodiment, the core is formed by granulating a mixture of trospium chloride or a pharmaceutically acceptable salt thereof with an excipient and milling to a desired particle size range. In another embodiment, the core can be formed by extrusion and spheronization of a mixture of trospium chloride or a pharmaceutically acceptable salt thereof with an excipient.

Granulating processes can include fluid bed granulation, wet granulation, hot melt granulation, and spray congealing. Other processes include slugging and roller compaction. The mixtures to be granulated can first be dry-blended. The dry-blended dry ingredients can be mixed with water before extrusion.

Extrusion and spheronization of a mixture of xanomeline or a pharmaceutically acceptable salt thereof and trospium chloride with an excipient provide desirable cores with a distribution of particle sizes as described herein and one or more other desirable properties. In certain embodiments, short processing times can lead to a more stable product. For example, reducing spheronization reduces friction and related heat, reducing the time the product is exposed to air (either when moist and/or before packaging) diminishes oxidation. On the other hand, rapid processing by extrusion and spheronization can lead to a poor-quality product, such as having a large fraction of the bead cores falling outside a desired particle size range. The moisture absorbed by spheronization aids (which happens over time) influences the beads' spheronization characteristics.

Accordingly, in one embodiment, the moisture content of the granulation mixture, before drying, ranging between about 20 wt. % and about 40 wt. %, such as between 25 wt. % and about 35 wt. %, between about 28 wt. % and about 32 wt. %, at least about 28 wt. %, at least about 28.5, between about 20 wt. % and about 40 wt. %, between about 25 wt. % and about 35 wt. %, between about 27 wt. % and about 31 wt. %, or between about 28.5 wt. % and about 31 wt. %.

In certain embodiments, the wet mass can be held before extrusion, allowing the spheronization aid to swell with granulating fluid. The hold time can be at least 15 minutes, such as at least 30 minutes, at least 45 minutes, or at least 60 minutes. In certain embodiments, the hold time ranges between about 15 minutes and about 120 minutes, such as between 30 and 100 minutes or between 60 and 90 minutes.

As described above relating to cores, the method can include a step of sorting (e.g., by sieving) the cores before optional coating to retain particles in a predetermined size range, for example, sizes ranging between about 0.7 mm and about 2.8 mm, such as between about 0.7 mm and about 2.5 mm, between about 0.8 mm and about 1.7 mm, or any range described herein.

As described above relating to beads, the method can include a step of sorting (e.g., by sieving) the beads after optional coating to retain particles in a size range, for example, sizes ranging between about 0.7 mm and about 2.8 mm, such as between about 0.7 mm and about 2.5 mm, or between about 0.8 mm and about 1.7 mm, or any range described herein.

In an extrusion and spheronization process, the following optional features can be employed, individually or in one or more combinations thereof. Water can be a granulation agent. Microcrystalline cellulose can be in the cores as a spheronization aid. Hypromellose can be included in the cores as a binder. The extrusion screen size can be 1.0 mm. The friction plate of the spheronizer can be cross-hatched. The friction plate of the spheronizer can be cross-hatched with a square pitch of at least about 3 mm, or greater than about 3 mm, or at least about 4 mm, or greater than about 4 mm, or ranging between about 3 mm and about 7 mm, or about 5 mm. The spheronization time can be less than about 5 minutes, or less than about 4 minutes, or less than about 3 minutes, or less than about 2 minutes, or up to 1 minute. The spheronized particles can include non-spherical particles (i.e., irregular shapes), for example, a substantial fraction thereof, such as at least about 20 wt. %, at least about 30 wt. %, at least about 40 wt. %, at least about 50 wt. %, at least about 60 wt. %, or at least about 70 wt. % thereof.

In certain embodiments, the pharmaceutical composition is stored with a desiccant, for example, pharmaceutical grades of silica gel, crystalline sodium, potassium or calcium aluminosilicate, colloidal silica, anhydrous calcium sulfate, and the like.

In certain embodiments, the pharmaceutical composition is stored with an oxygen absorber.

In certain embodiments, the pharmaceutical composition is stored under a dry inert gas such as nitrogen, helium, argon, neon, xenon, krypton, or a mixture thereof.

In certain embodiments, the pharmaceutical composition is stored under reduced pressure compared to the external ambient air.

In certain embodiments, the pharmaceutical composition is stored at a reduced temperature, e.g., at refrigerated temperatures (e.g., 2° C. to 8° C.). In certain embodiments, the pharmaceutical composition is stored in such a manner that has fewer impurities, such as Impurity A, than when stored at 25° C.

In certain embodiments, the pharmaceutical composition is stored by a manufacturer, a distributor, a pharmacy, or a hospital at a temperature of between about 2° C. and about 8° C. before dispensing the oral pharmaceutical composition to the subject. In certain embodiments, after the oral pharmaceutical composition is dispensed to the subject, the pharmaceutical composition is stored at a temperature of between about 20° C. and about 25° C.

Also provided is a method of stabilizing a pharmaceutical dosage form or composition described herein, comprising storing the dosage form at a temperature of about 2° C. to about 8° C.

In certain embodiments, a method for preparing a pharmaceutical dosage form comprising xanomeline beads comprises forming a wet mass comprising xanomeline tartrate and an excipient, optionally microcrystalline cellulose, with a moisture content ranging between about 20 wt. % and about 40 wt. %, extruding and spheronizing the wet mass comprising xanomeline tartrate and excipient to make cores, sorting the cores to a target particle size range, optionally between about 0.7 mm and about 2.5 mm, coating the sorted cores with a polymer to form beads comprising a core and a coating, and sorting the bead particles to a target particle size range, optionally between about 0.7 mm and about 2.5 mm.

In certain embodiments, a method for preparing a pharmaceutical dosage form comprising trospium beads comprises forming a wet mass comprising trospium chloride and an excipient, optionally microcrystalline cellulose, with a moisture content ranging between about 20 wt. % and about 40 wt. %, extruding, spheronizing, and drying the wet mass comprising trospium chloride and excipient to make cores, sorting the cores to a target particle size range, optionally between about 0.7 mm and about 2.5 mm, coating the sorted cores with a polymer to form beads comprising a core and a coating, and sorting the bead particles to a target particle size range, optionally between about 0.7 mm and about 2.5 mm.

### Purity

Also provided is the compound 3-[(4-hexyloxy)-1,2,5-thiadizaol-3-yl]-5-hydroyl-1-methylpyridin-1-ium.

Also provided is a pharmaceutical composition, comprising xanomeline and/or a salt thereof and less than 0.5 wt. % 3-[(4-hexyloxy)-1,2,5-thiadizaol-3-yl]-5-hydroxyl-1-methylpyridin-1-ium (Impurity A). In certain embodiments, the pharmaceutical composition comprises less than 0.30 wt. % of Impurity A, such as less than 0.25 wt. %, less than 0.20 wt. %, less than 0.15 wt. %, less than 0.14 wt. % or less than 0.1 wt. %. Also provided is a pharmaceutical composition, comprising xanomeline and/or a salt thereof and less than 0.15 wt. % 3-[(4-hexyloxy)-1,2,5-thiadizaol-3-yl]-5-hydroyl-1-methylpyridin-1-ium (Impurity A).

Also provided is an oral pharmaceutical composition, comprising a plurality of xanomeline beads comprising xanomeline or a salt thereof and less than 0.5 wt. % 3-[(4-hexyloxy)-1,2,5-thiadizaol-3-yl]-5-hydroxyl-1-methylpyridin-1-ium; and a plurality of trospium beads comprising a salt of trospium. Also provided is an oral pharmaceutical composition, comprising a plurality of xanomeline beads comprising xanomeline or a salt thereof and less than 0.15 wt. % 3-[(4-hexyloxy)-1,2,5-thiadizaol-3-yl]-5-hydroxyl-1-methylpyridin-1-ium; and a plurality of trospium beads comprising a salt of trospium.

In certain embodiments, the pharmaceutical composition comprises less than 0.5 wt. % of Impurity A after the pharmaceutical composition is stored for at least 3 months at 40° C. and 75% relative humidity.

In certain embodiments, the total impurities in the pharmaceutical compositions provided herein are no greater than about 5% by weight, no greater than about 4% by weight, no greater than about 3% by weight, no greater than about 2.5% by weight, no greater than about 2% by weight, no greater than about 1.5% by weight, no greater than about 1% by weight, no greater than about 0.5% by weight, or no greater than about 0.1% by weight.

### EXAMPLES

The following examples are provided for illustration and are not intended to limit the scope of the disclosure.

### Example 1—A Phase 1, Randomized, Double-Blind, Multiple-Dose Study Evaluating the Safety, Tolerability, and Exploratory PK of KarXT in Healthy Elderly Subjects

The purpose of this Phase 1b clinical trial was to evaluate the safety and tolerability of KarXT in healthy elderly volunteers and to assess its potential efficacy and safety in dementia-related psychosis (DRP). In the Phase 1b trial, the various doses and ratios were evaluated for both xanomeline and trospium in healthy elderly volunteers using a flexible dosing protocol titrated over about 2 to 3 weeks. The maximum dose was reached on Day 12, 15, and 17 in Cohorts 1, 2, and 3, respectively. Dosing lasted a total of 14, 18, and 20 days, respectively. Each cohort consisted of 16 healthy elderly volunteers.

This was a Phase 1, randomized, double-blind, multiple-dose, adaptive-design study evaluating the safety, tolerability, and exploratory PK of KarXT in healthy elderly subjects. The study consisted of a screening period of up to 28 days. This inpatient investigative period included a treatment period and a post-treatment observation period. A follow-up visit was completed 7±3 days after administering the final dose of the study drug. For Cohort 1, the inpatient investigative period was up to 17 days, including admission, a treatment period of up to 14 days, and a 2-day post-treatment observation period. For Cohort 2, the inpatient investigative period was up to 21 days, including admission, a treatment period of up to 18 days, and a 2-day post-treatment observation period. For Cohort 3, the inpatient investigative period was up to 23 days, including admission, a treatment period of up to 20 days, and a 2-day post-treatment observation period.

Subjects were randomized 3:1 in each planned cohort to receive KarXT (administered as a combined formulation of xanomeline tartrate and trospium chloride, or coadministration of individual xanomeline tartrate [KarX] and trospium chloride [KarT] capsules) or placebo.

The study drug was an oral capsule of xanomeline tartrate/trospium chloride (KarXT). For Cohorts 1 and 2, the study drug strength was xanomeline tartrate 25 mg and trospium chloride 10 mg (KarXT 25/10 mg), KarXT 50/10 mg, KarXT 75/10 mg, and KarXT 75/20 mg. For Cohort 3, xanomeline and trospium was coadministered as individual KarX and KarT capsules. The dose of each could be adjusted individually, based on the following available dose strengths: KarX (xanomeline tartrate) 25 mg and 50 mg and KarT (trospium chloride) 5 mg and 7.5 mg.

The placebo visually matched the active medication. For Cohorts 1 and 2, microcrystalline cellulose was packed in an opaque white hydroxypropyl methylcellulose capsule. For Cohort 3, suglets and talc in size 0 were packed in a Swedish orange opaque hard-shell capsule with no markings.

In each planned cohort, a sentinel group of four subjects was dosed (3 active:1 placebo), and their data were reviewed before dosing the remaining subjects. Dosing of the remaining twelve subjects commenced once the study drug's safety and tolerability were confirmed in the sentinel dosing group upon completing the treatment. A maximum of three subjects per cohort were aged 60 years to 64 years, and only one subject in this age range could be in the sentinel dosing group.

Subjects assigned to active treatment in Cohorts 1 and 2 started treatment with a lead-in dose of KarXT for 2 days. Subjects assigned to active treatment in Cohort 3 started treatment with a lead-in dose of KarXT for 4 days. KarXT doses were increased beginning on Day 3 (or Day 5 for Cohort 3) according to cohort-specific plans (FIGURE S 1) to achieve the following final cohort-specific total daily dose (TDD) of KarXT, if tolerated:
Cohort 1: 200/40 mg
Cohort 2: 200/30 mg
Cohort 3: 150/20 mg

After each cohort completed, the timing of the dose titration steps could be adjusted, and/or the dose strengths at a given titration step could be lowered based on the safety and tolerability of KarXT administered in previous cohorts.

The main inclusion criteria were males and postmenopausal females, aged 60 years to 85 years, including the screening. The body mass index was 18.0 kg/m² to 35.0 kg/m², inclusive. The included subjects had good general health, in the opinion of the study investigator.

Exclusion criteria included a history or presence of clinically significant cardiovascular, pulmonary, hepatic, renal, hematologic, GI, endocrine, immunologic, dermatologic, neurologic, oncologic, or psychiatric disease other than the following conditions which must have been controlled with medical treatment for at least three months before dosing: osteoarthritis, osteopenia, type II diabetes mellitus, hypercholesterolemia, hypertension, hypothyroidism, hyperthyroidism, and vitamin B₁₂ deficiency. Other chronic conditions were allowed if stable for at least three months before screening.

Subjects were also excluded if they had a history of syncope, presyncope or symptomatic hypotension, or symptomatic orthostatic hypotension at screening or admission, irritable bowel syndrome (with or without constipation) or serious constipation requiring treatment within the last six months, a history of or high risk for urinary retention (including subjects with a history of symptomatic benign prostatic hyperplasia for Cohort 2 and subsequent cohorts), gastric retention, or narrow-angle glaucoma (also known as angle-closure glaucoma). For male subjects, a score of >4 on the International Prostate Symptom Score (I-PSS) at Screening and/or Baseline was exclusionary. Also exclusionary were a history of alcohol or drug abuse within the last year or current abuse as determined by urine drug screen or breathalyzer test at screening, significant liver function test abnormality, major depressive episode within the past year, significant suicidal ideation or any attempt, or any other condition that, in the opinion of the Investigator, would jeopardize the safety of the subject or the validity of the study results clinically significant abnormal finding on physical examination, ECG, vital signs, or clinical laboratory results at screening, including QTcF >450 ms for males and >470 ms for females. An estimated glomerular filtration rate (eGFR) below 60 mL/min/1.73 m² using the Modification of Diet in Renal Disease Study equation is a specific exclusion. Also exclusionary were ALT, ALP, AST, or TB outside the normal range at baseline, having received any investigational agent within 30 days before dosing or within five half-lives, whichever is longer, the use of any prescription medication within 14 days before enrollment except hormone replacement treatment for postmenopausal syndrome and those used to treat allowed conditions. Such medications must have been at a stable dose for at least three months before dosing and must be anticipated to remain stable for the study duration. Subjects with a previous positive test for HIV-1 or -2, hepatitis B or C, or a positive test for these conditions were obtained at the screening.

Oral or inhaled steroids used other than on an as-needed basis within one year before screening, the use of vitamins, herbs, supplements, or over-the-counter (OTC) medications, except for calcium, vitamin D, low-dose aspirin, glucosamine, and chondroitin, are excluded within two weeks before enrollment, and during the entire duration of the study. Zofran was allowed for treating nausea, and acetaminophen and/or ibuprofen were allowed during the study. The use of vitamin B12 for vitamin B12 deficiency was allowed. Patients were excluded if smoking cessation products, such as varenicline, were used within 30 days before screening.

The assessment of adverse events, clinical laboratory samples, electrocardiograms (ECGs), vital signs (including orthostatic vital signs), urine output monitoring, Columbia-Suicide Severity Rating Scale (C-SSRS), serial PK sampling, and collecting saliva to measure saliva volume were performed at defined time points during the study.

The data from Cohorts 1 and 2 demonstrated that lower oral doses of both drugs, xanomeline, and trospium, resulted in higher blood levels in healthy elderly subjects than adults under 65. Furthermore, oral doses of xanomeline between about 100 and 200 mg per day in the healthy elderly volunteers resulted in mean plasma levels (Cₘₐₓ) comparable to, or slightly higher than the blood levels observed in EMERGENT-1, the completed positive Phase 2 trial which studied doses of 200-250 mg of xanomeline and demonstrated robust antipsychotic efficacy in adults with schizophrenia. (See U.S. Pat. Nos. 10,925,832 and 10,933,020, and U.S. Ser. No. 16/950,203, incorporated herein by reference in their entireties.)

Additionally, data from the completed cohorts have suggested that a lower ratio of trospium to xanomeline, compared to the ratios used in adults under the age of 65, was better tolerated by the healthy elderly subjects. The cholinergic and anticholinergic adverse events (AEs) seen in Cohorts 1, 2 and 3 were similar to those observed in prior trials of KarXT, with the vast majority (>80%) categorized as mild.

Referring to Table 1, one drug-related serious adverse event (SAE) was reported in Cohort 1. The SAE was urinary retention in a subject receiving a 40 mg total daily dose of trospium. No cases of syncope were observed in any cohort. There were no SAEs reported in cohort 2 and 3.

TABLE 1
Adverse Events

| Selected AE data | | | |
|---|---|---|---|
| (% of subjects) | Cohort 1 | Cohort 2 | Placebo |
| Constipation | 67% | 25% | 25% |
| Dry mouth | 50% | 17% | 0% |
| Urinary retention | 17% | 0% | 0% |
| SAEs | 8% | 0% | 0% |
| No AEs or mild AEs only | 33% | 58% | 100% |
| % of AEs rated mild | 81% | 87% | 100% |

The frequency and severity of trospium-related side effects using 40 mg TDD of trospium was unexpected, which was the low dose in the previous schizophrenia trial (40-60 mg TDD) and the approved dose of trospium for treating overactive bladder. Reducing the trospium dose reduced trospium adverse events, including constipation reducing from 42% to 0% placebo adjusted, dry mouth from 50% to 17% placebo adjusted, urinary retention from 17% to 0%. Serious adverse events were reduced from 1 to 0.

Surprisingly, reducing the trospium dose, thereby using a xanomeline:trospium ratio higher than studied in normal healthy volunteers and patients with schizophrenia, improved the overall tolerability for KarXT, which depended on the balance of xanomeline and trospium. This improvement was demonstrated by reduced overall adverse event severity. No serious or severe adverse events occurred in Cohort 2. The percentage of patients who experienced either no adverse events or only mild adverse events on KarXT increased by 25%. In certain embodiments, the dose for elderly subjects should differ from schizophrenia or normal healthy volunteers. Whereas a dose of between 40 and 60 mg trospium chloride TDD was successful used in patients with normal healthy volunteers and patients living with schizophrenia, a dose of 30 mg trospium chloride TDD performed better than 40 mg TDD in normal elderly volunteers. Testing of 60 mg TDD was not completed in the elderly based on the AEs observed with 40 mg TDD. This relationship can also be expressed as ratios between xanomeline and trospium, where a ratio of 5:1 xanomeline/trospium or lower was used in the normal healthy volunteers and patients living with schizophrenia (200 mg xanomeline with 40 mg trospium, a 5:1 ratio; 250 mg xanomeline with 60 mg trospium, a 4.2:1 ratio). This same 5:1 xanomeline:trospium ratio led to undesirable trospium related tolerability concerns in the elderly, where was ratios of greater than 5:1 xanomeline/trospium, or 6:1 xanomeline:trospium, or 7.5:1 xanomeline/trospium were better tolerated. In certain embodiments, the ratio of xanomeline to trospium is greater than 5:1, such as greater than 6:1, greater than 7.5:1, or greater than 10:1. In certain embodiments, the ratio of xanomeline to trospium is 10:1.

The subject with the serious adverse event of urinary retention was age 66. As noted above, all subjects were screened for estimated globular filtration rate (eGFR), and any patient with an EGFR less than 45 was excluded from the trial. The subjects with urinary retention, including the subject references above with an SAE of urinary retention, had eGFR values of 65 and 67, respectively, and all subjects had eGFR values of 54 or greater and thus, would not be expected to have creatinine clearance less than 30 mL/min.

Table 2 contains the xanomeline Cₘₐₓ data collected on the final day of the dosing period for cohorts 1, 2, and 3 in the healthy elderly trial compared to day 28 (last day PK data was collected) in the EMERGENT-1 trial for a patient population of ages 18-60 inclusive.

TABLE 2
Pharmacokinetics Data of Xanomeline

| | | | | 125/30 mg |
|---|---|---|---|---|
| | | | | dose group |
| | Cohort 1 | Cohort 2 | Cohort 3 | in EMERGENT-1 |
| mean Cₘₐₓ (ng/ml) | 7.3 ± 2.0 | 14.2 ± 5.3 | 7.2 ± 2.0 | 9.4 ± 1.5 |
| Average dose given before PK | 67 | 61 | 39 | 125 |
| measurements (mg) | | | | |
| Average total daily dose at | 172 | 156 | 116 | 250 |
| the end of the study (mg) | | | | |

Even though the average total daily dose of xanomeline at the end of the trial in cohort 2 was only 156 mg, the reported xanomeline Cₘₐₓ was higher in the elderly subjects than the patient with schizophrenia. Given that a potent antipsychotic effect was observed in the schizophrenia trial, these data suggested that xanomeline doses in the elderly between 100 and 200 mg could provide xanomeline blood levels equivalent or higher. Thus, these doses should be the therapeutically effective amount to produce an antipsychotic benefit in an elderly population with psychotic symptoms, such as dementia-related psychosis.

Analysis of the Cohort 3 data singly and combined with the data from Cohorts 1 and 2 confirmed the findings of the preliminary analysis. For example, the report of urinary retention was related to a higher dose of trospium used in Cohort 1 than doses used in Cohorts 2 and 3, where urinary retention was not observed. No serious or severe AEs were observed in Cohorts 2 and 3.

### Example 2—A Phase 3, Double-Blind, Placebo-Controlled Relapse Prevention Study to Evaluate the Safety and Efficacy of KarXT for the Treatment of Psychosis Associated with Alzheimer's Disease Dementia

This study evaluates the treatment of psychosis and prevention of relapse in subjects with psychosis associated with AD dementia treated with KarXT or placebo. This study also aims to evaluate the time from randomization to discontinuation for any reason in these subjects, to evaluate the safety and tolerability of KarXT compared with placebo, to evaluate the efficacy of KarXT compared with placebo during the Double-Blind Randomized Withdrawal Treatment Period and for KarXT during the Single-blind Treatment Period using the Neuropsychiatric Inventory Clinician (NPI-C) psychosis score (hallucinations and delusions), NPI-C Core score, CGI-S scale, Clinical Global Impression-Improvement (CGI-I) scale, Cohen-Mansfield Agitation Inventory (CMAI), NPI-C Total score and Mini-Mental State Examination (MMSE), to evaluate the PK of KarXT in subjects with psychosis associated with AD dementia.

This study will be conducted as a phase 3 randomized withdrawal study of KarXT in subjects with psychosis related to AD dementia. Subjects with AD dementia must also have symptoms of agitation or aggression, common symptoms in AD dementia psychosis.

Male and female subjects aged 55-90 years having mild to severe AD dementia with moderate to severe psychosis will be enrolled in this study.

Individuals must meet all of the following criteria to be included in the study:
Is a male or female aged 55 to 90 years, inclusive, at Screening (Visit 1A)
Meets clinical criteria for possible or probable AD
Has a Magnetic Resonance Imaging (MRI) or Computed Tomography (CT) scan of the brain (completed within the past five years) taken during or after the onset of dementia to rule out other central nervous system (CNS) diseases that could account for the dementia syndrome, e.g., major stroke, neoplasm, subdural hematoma.
History of psychotic symptoms meeting International Psychogeriatric Association (IPA) criteria for at least two months before Screening (Visit 1A).

See Table 3 of Cummings J, Pinto L C, Cruz M, et al. “Criteria for Psychosis in Major and Mild Neurocognitive Disorders: International Psychogeriatric Association (IPA) Consensus Clinical and Research Definition.” Am J Geriatr Psychiatry. 2020 December; 28(12):1256-1269, incorporated herein by reference in its entirety.
CGI-S scale with a score ≥4 (moderate) at Screening (Visit 1A)
AD dementia subjects are required to have at Screening (Visit 1A):
a. Moderate to severe delusions, defined as NPI-C Delusion domain score of ≥2 on two of the eight items
b. NPI Agitation/Aggression domain score ≥4
MMSE score of 8 to 22, inclusive, at Screening (Visit 1A)
If the subject is taking a cholinesterase inhibitor and/or memantine, they must have been on a stable dose for six weeks before screening (Visit 1A) and be willing to maintain a stable dose for the duration of the study.

Subjects will be excluded from the study if one or more of the following criteria at Screening are applicable:
Psychotic symptoms primarily attributable to a condition other than the AD causing dementia, e.g., schizophrenia, schizoaffective disorder, delusional disorder, or mood disorder with psychotic features
History of a major depressive episode with psychotic features during the 12 months before screening (Visit 1A)
History of an axis I diagnosis of delirium, amnestic disorder, bipolar disorder, schizophrenia, or schizoaffective disorder
Significant or severe medical conditions including pulmonary, hepatic, renal, hematologic, gastrointestinal, endocrine, immunologic, dermatologic, neurologic, or oncologic disease or any other condition that could jeopardize the safety of the subject, ability to complete or comply with the study procedures or validity of the study results
History of ischemic stroke within 12 months before screening (Visit 1A) or any evidence of hemorrhagic stroke
History of cerebral amyloid angiopathy (CAA), epilepsy, central nervous system (CNS) neoplasm, unstable thyroid function, or unexplained syncope
Any of the following:
a. New York Heart Association (NYHA) Class 2 congestive heart failure
b. Grade 2 or greater angina pectoris
c. Sustained ventricular tachycardia
d. Ventricular fibrillation
e. Torsade de pointes
f. Implantable cardiac defibrillator
Myocardial infarction within the six months before screening (Visit 1A)
Personal or family history of symptoms of long QT syndrome
Human immunodeficiency virus (HIV), cirrhosis, biliary duct abnormalities, hepatobiliary carcinoma, and/or active hepatic viral infections as indicated by medical history or LFT results
History or high risk of urinary retention, gastric retention, or narrow-angle glaucoma
History of irritable bowel syndrome (with or without constipation) or serious constipation requiring treatment within the last six months
Risk of suicidal behavior during the study as determined by the Investigator's clinical assessment and/or C-SSRS as confirmed by the following:
a. Answers “Yes” on items 4 or 5 (C-SSRS—ideation) with the most recent episode occurring within the two months before screening or,
b. Answers “Yes” to any of the five items (C-SSRS behavior) with an episode occurring within the 12 months before screening
Recent history of receiving monoamine oxidase inhibitors, anticonvulsants (e.g., lamotrigine, divalproex), lithium, tricyclic antidepressants (e.g., imipramine, desipramine), or any other psychoactive medications except for as-needed anxiolytics (e.g., lorazepam, chloral hydrate)
a. Selective serotonin reuptake inhibitors and serotonin-norepinephrine reuptake inhibitors taken at a stable dose for at least eight weeks before screening (Visit 1A) may be permitted
b. Mirtazapine may be used as a hypnotic if started at least eight weeks before screening (Visit 1A)

Subjects will receive single-blind KarXT for during the Single-blind Treatment Period. Each subject will be titrated to the maximum dose of single-blind KarXT suitable for the subject based on tolerability and efficacy, up to a maximum of 200 mg xanomeline/20 mg trospium/day. Dosing is flexible; however, subjects will need to reach a minimum dose of KarXT 90/9 mg/day by the end of Week 6 to stay in the study.

KarXT is expressed as mg xanomeline tartrate salt/mg trospium chloride, available in oral capsules: KarXT 20/2.0 mg, KarXT 30/3.0 mg, KarXT 40/4.0, KarXT 50/5.0 mg, and KarXT 66.7/6.67 mg. Capsules for each dosage strength were produced with xanomeline beads (Table 3) and either 4.4 wt. % or 17.7 wt. % trospium beads (Table 4), depending on the dosage strength (Table 5). Specifically, for dosages with 5.0 mg trospium chloride or less, beads with a drug loading of 4.4% trospium chloride were used. KarXT 66.7/6.67 mg used trospium beads with 17.7% trospium chloride. All dosage forms conformed on appearance of the capsules, identity by retention time (RT), identity by ultraviolet spectrum, dissolution (at least 80% at 30 min for each xanomeline and trospium), and content uniformity (per USP <905). The dosage forms also conformed to the specifications for the assay, impurities and related substances, water content, and microbial limits (Table 6).

TABLE 3
Xanomeline Bead Formulation

| Ingredient | wt. % |
|---|---|
| Xanomeline tartrate | 66 |
| Microcrystalline cellulose | 33 |
| (Avicel Ph-101, NF/EP) | |
| Ascorbic acid | 0.5 |
| Purified Water, USP* | 22.3 |
| Talc, USP/ EP | 0.5 |
| Total | 100 |

*Evaporated during processing

TABLE 4
Trospium Bead Formulation

| Ingredient | wt. % | wt. % |
|---|---|---|
| Trospium Chloride | 4.4 | 17.7 |
| Avicel PH -101, NF/EP | 54.5 | 46.8 |
| Lactose, USP/NF/EP | 40.6 | 35 |
| Purified Water, USP* | 60 | 46 |
| Talc, USP/EP | 0.5 | 0.5 |
| Total | 100 | 100 |

*Evaporated during processing

TABLE 5
Encapsulations

| | Dosage Strength Xanomeline Tartrate mg/Trospium Chloride mg | | | | | |
|---|---|---|---|---|---|---|
| | Placebo | 20/2 | 30/3 | 40/4 | 50/5 | 66.7/6.67 |
| Capsule Size | size 2 | size 2 | size 2 | size 2 | size 2 | size 2 |
| Capsule type | Coni Snap V Caps Plus, Swedish Orange Opaque/Swedish Orange Opaque Imp | | | | | |
| | N/A | 4.4% Trospium Cl | 4.4% Trospium Cl | 4.4% Trospium Cl | 4.4% Trospium Cl | 17.7% Trospium Cl |
| | | beads | beads | beads | beads | beads |

* N/A = Not applicable

TABLE 6
Tests on encapsulated dosages.

| | | Dosage Strength | | | | |
|---|---|---|---|---|---|---|
| Test | | 20/2 | 30/3 | 40/4 | 50/5 | 66.7/6.67 |
| LC Assay (90.0-110.0% | Xanomeline | 94.5% | 96.1% | 100.2% | 97.6% | 99.1% |
| for each API) | Trospium | 100.2% | 99.1% | 103.8% | 98.7% | 97.8% |
| Impurities and Related | Unspecified Individual ≤0.2% LC | <LOQ | <LOQ | <LOQ | <LOQ | <LOQ |
| Substances | Total Impurities ≤1.5% LC | 0.0% | 0.0% | 0.0% | 0.0% | 0.0% |
| | Trospium Cl RC B ≤0.2% LC | ND | ND | ND | ND | ND |
| | Trospium Cl RC A ≤0.2% LC | ND | ND | ND | ND | ND |
| | Xanomeline hydroxypyridinium | ND | ND | ND | ND | ND |
| | (RRT 1.09) ≤0.2% LC | | | | | |
| | Xanomeline N-Oxide | ND | ND | ND | ND | ND |
| | (RRT 1.10) ≤0.5% LC | | | | | |
| Water content | Result reported | 2.2% | 1.7% | 1.9% | 1.7% | 2.0% |
| Microbial limits | Total yeasts and mold counts ≤100 cfu/g | <8 cfu/g | <8 cfu/g | <8 cfu/g | <8 cfu/g | <8 cfu/g |
| | Total aerobic microbial counts ≤1000 cfu/g | <8 cfu/g | <8 cfu/g | <8 cfu/g | <8 cfu/g | <8 cfu/g |
| | E. coli: Absent | Absent | Absent | Absent | Absent | Absent |

* LOQ = limit of quantification;
ND = Not detectable

In the absence of tolerability issues, subjects should have the dose increased if efficacy has not been observed. On Day 1, the subject will take one dose of study medication in the evening.
Day 1: Subjects will start on KarXT 60/6 mg/day (20/2.0 mg TID)
End of Week 1: Dose will be increased to 90/9 mg/day (30/3.0 mg TID)
End of Week 2: Dose should be increased to 120/12 mg/day (40/4.0 mg TID) unless the subject is experiencing tolerability issues
End of Week 3: Optional dose increase to 150/15 mg/day (50/5.0 mg TID)
End of Week 4: Optional dose increase to 200/20 mg/day (66.7/6.67 mg TID)
Weeks 5 onward: Continue dosing as per End of Week 5 or modify as per subject's tolerability and clinical response

Subjects must meet the following criteria at the end of the Single-blind Treatment Period before randomization and administration of the first dose of study drug in the Double-blind Randomized Withdrawal Period:
1. Meet the following response criteria
   a. ≥40% decrease (an improvement from Baseline at the end of the single blind treatment period (start of the Double-blind Randomized Withdrawal Treatment Period) on the NPI-C H+D score AND
   b. CGI-I score of 1 (very much improved) or 2 (much improved). The CGI-I requires the assessor to consider aspects of the agitation/aggression and psychosis before providing a global assessment of change. These aspects include emotional or psychomotor agitation, verbal or physical aggression, delusions, and hallucinations.
2. Taking a minimum KarXT dose of 90/9 mg/day (30/3.0 mg TID) 12 (Visit 10)

The responder and relapse definitions will be based on the NPI-C Psychosis score (the sum of hallucination and delusion subscores).

The Treatment Period is based on hallucinations and delusion domains, two domains of the NPI-C, a 14-domain scale, and the CGI-I scale. Each domain on the NPI-C scale has multiple items. Each item has a Clinical Impression severity score of 0-3. This study defines the NPI-C H+D score (maximum score=45 points) contains the two domains. The delusion domain has 8 items (maximum score=24), and hallucination domain has 7 items (maximum score=21).

The response criteria for entry into the Double-blind Randomized Withdrawal Treatment Period are a ≥40% decrease (improvement) at the start of the Double-blind Randomized Withdrawal Treatment Period on the NPI-C H+D score compared to baseline (Day 1) and a CGI-I score of 1 (very much improved) or 2 (much improved) at the start of the double-blind randomized withdrawal treatment period. The CGI-I requires the assessor to consider aspects of the agitation/aggression and psychosis before providing a global assessment of change. These aspects include emotional or psychomotor agitation, verbal or physical aggression, delusions, and hallucinations.

The primary endpoint of the study is the time from randomization to relapse during the Double-blind Randomized Withdrawal Treatment Period. A subject must meet at least one of the following four criteria to be considered to have relapsed. A ≥40% increase (worsening) from the start of Double-blind Randomized Withdrawal Treatment Period) on the NPI-C H+D score and a CGI-I score of 6 (much worse) or 7 (very much worse) relative to the start of the Double-blind Period, with both criteria met at the same visit. The CGI-I requires the assessor to consider aspects of the agitation/aggression and psychosis before providing a global assessment of change. The single blinded treatment period can be 8, 10, 12, 14 or 16 weeks and the double-blind treatment period can be 22, 24, 26, 28, or 30 weeks.

A subject must meet at least one of the following four criteria to be considered to have relapsed:
≥40% increase (worsening) from the start of the Double-blind Randomized Withdrawal Treatment Period) on the NPI-C H+D score AND a CGI-I score of 6 (much worse) or 7 (very much worse) relative to the start of Double-blind Randomized Withdrawal Treatment Period, with both criteria met at the same visit. These aspects include emotional or psychomotor agitation, verbal or physical aggression, delusions, and hallucinations.
Subject is treated with an antipsychotic (other than KarXT) for dementia-related delusions and/or hallucinations within the Double-blind Randomized Withdrawal Treatment Period.
Subject stops study drug or withdraws from the study for lack of efficacy (as reported by the subject or caregiver/study partner) or Investigator discontinues study drug due to lack of efficacy during the Double-blind Randomized Withdrawal Treatment Period.
Subjects hospitalized for worsening psychosis, agitation, or aggression symptoms during the Double-blind Randomized Withdrawal Treatment Period.

The exploratory efficacy endpoints include:
Change from baseline to the end of the Single-blind Treatment Period on the NPI-C: H+D score, NPI-C Core score (hallucinations, delusions, agitation, and aggression domains), CGI-S, CMAI, NPI-C Total score, and MMSE. CGI-S requires the assessor to consider aspects of the psychosis prior to providing a global assessment of change. These aspects include hallucinations and delusions.
Change from randomization to endpoint on the NPI-C: H+D score, NPI-C Core score (hallucinations, delusions, agitation, and aggression domains), CGI-S, CMAI, NPI-C Total score and MMSE.
CGI-I score at the end of the single blind treatment period and endpoint. CGI-I requires the assessor to consider aspects of the psychosis prior to providing a global assessment of change. These aspects include hallucinations and delusions.
Treatment response (NPI-C: H+D score ≥40% decrease and CGI-I score of 1 [very much improved] or 2 [much improved] at the end of the Single-blind Treatment Period and 2 week prior) Treatment response (NPI-C Core: H+D score ≥40% decrease and CGI-I score of 1 [very much improved] or 2 [much improved])] at the Week 10 Visit and the end of the 12-week Single-blind Treatment Period.)

### Example 3—A Phase 3, Double-Blind, Placebo-Controlled, Parallel Group Study to Evaluate the Safety and Efficacy of KarXT for the Treatment of Psychosis Associated with Alzheimer's Disease Dementia

The purpose of this study is to evaluate the efficacy of KarXT compared with placebo in the treatment of subjects with psychosis associated with AD as measured by the NPI-C Psychosis score (hallucinations and delusions), score and to evaluate the efficacy of KarXT compared with placebo for treating subjects with psychosis associated with AD as measured by the CMAI. This study also aims to evaluate the efficacy of KarXT compared with placebo on the NPI-C Core score, NPI-C Agitation/Aggression score (agitation and aggression domains), NPI-C Total score, and CGI-I score to assess the impact of KarXT on cognition using the Mini-Mental State Examination (MMSE), to characterize the pharmacokinetics (PK) of KarXT in subjects with psychosis associated with AD dementia.

Male and female subjects aged 55-90 years and have mild to severe AD dementia with moderate to severe psychosis will be enrolled in this study. The exclusion and inclusion criteria are the same as Example 2.

The primary endpoint is a change from baseline on the NPI-C H+D score, containing the two domains of hallucinations and delusions. The delusion domain has 8 items (maximum score=24), and the hallucination domain has 7 items (maximum score=21). The full NPI-C core score could also be used.

Subject may receive placebo during a lead period and subjects with an NPI-H+D score reduction of 40% (change from baseline to the end of Week 2) or more will not be randomized. In addition, subjects must have a CGI-S score ≥4 (moderate), NPI-C Delusion domain score of ≥2 on two of the eight items, and NPI Agitation/Aggression domain score ≥4. The NPI Agitation/Aggression domain score ≥4 has been used in two brexpiprazole and one escitalopram agitation in AD subject studies as an inclusion criterion.

Subjects will receive double-blind KarXT or placebo during the Randomized Treatment Period, which in some examples last 12 weeks. Each subject will be titrated to the maximum dose of KarXT suitable for the subject based on tolerability and efficacy, up to a maximum of 200/20 mg/day. Dosing is flexible; however, subjects will need to reach a minimum dose of KarXT 120/12 mg/day by the End of Week 6.

KarXT is expressed as mg xanomeline as the tartrate salt/mg trospium chloride in oral capsules: KarXT 20/2.0 mg, KarXT 30/3.0 mg, KarXT 40/4.0 mg, KarXT 50/5.0 mg, and KarXT 66.7/6.67 mg. The dosage forms described in Example 2 will also be used in this study. Administration of KarXT will be flexible from Week 3 of treatment based on tolerability and clinical response.
Day 1: Subjects will start on KarXT 60/6 mg/day (20/2.0 mg TID)
End of Week 1: Dose will be increased to 90/9 mg/day (30/3.0 mg TID)
End of Week 2: Dose should be increased to 120/12 mg/day (40/4.0 mg TID) unless there are experiencing tolerability issues
End of Week 3: Optional dose increase to 150/15 mg/day (50/5.0 mg TID)
End of Week 4: Optional dose increase to 200/20 mg/day (66.7/6.67 mg TID)
Weeks 5 onward: Continue dosing as per End of Week 5 or modify as per subject's tolerability and clinical response

The primary efficacy endpoint is the change from baseline to endpoint in the NPI-C H+D Score, analyzed using a mixed model for repeated measures (MMRM) with an unstructured variance-covariance matrix, with fixed-class effect terms for treatment, trial center and visit a week, an interaction term of treatment by visit week, and the interaction term of baseline NPI-C H+D score by visit week as a covariate. The key secondary primary efficacy endpoint is the change from baseline to endpoint in CMAI Total score, analyzed using an MMRM with an unstructured variance-covariance matrix, with fixed-class effect terms for treatment, trial center and visit a week, an interaction term of treatment by visit week, and the interaction term of baseline CMAI Total score by visit week as a covariate.

Secondary efficacy analyses include the following in order:
Change from baseline to endpoint on the NPI-C Psychosis score, NPI-C Core score, NPI-C Agitation, and NPI-C Aggression scores, and NPI-C Total score
Change from baseline to endpoint on the CGI-S related to psychosis, agitation, and aggression
Percentage of subjects with 50% decrease from baseline to endpoint on the NPI-C Core score at endpoint
CGI-I related to psychosis, agitation, and aggression at the endpoint.

### Example 4

The purpose of this study is to evaluate the efficacy of KarXT compared with placebo in the treatment of subjects with psychosis associated with AD as measured by the NPI-C H+D score and to evaluate the efficacy of KarXT compared with placebo for treating subjects with psychosis associated with AD as measured by the CMAI. This study also aims to evaluate the efficacy of KarXT compared with placebo on the NPI-C Core score, NPI-C Agitation/Aggression score (agitation and aggression domains), NPI-C Total score, and CGI-I score to assess the impact of KarXT on cognition using the Mini-Mental State Examination (MMSE), to characterize the pharmacokinetics (PK) of KarXT in subjects with psychosis associated with AD dementia.

Male and female subjects aged 55-90 years and have mild to severe AD dementia with moderate to severe psychosis will be enrolled in this study. The exclusion and inclusion criteria are the same as Example 2.

The primary endpoint is a change from baseline on the NPI-H+D score, containing the two domains of hallucinations and delusions. The delusion domain has 8 items (maximum score=24) and the hallucination domain has 7 items (maximum score=21).

For the first twelve weeks, subjects will be divided into two cohorts. The first cohort will be treated with KarXT, and the second cohort treated with placebo. Improvements in NPI-C H+D are recorded between the first and second cohorts. Each subject in the first cohort will be titrated to the maximum dose of KarXT suitable for the subject based on tolerability and efficacy, up to a maximum of 200/20 mg/day. Dosing is flexible; however, subjects will need to reach a minimum dose of KarXT 120/12 mg/day by the End of Week 12.

Improvement will be measured as an increase in an NPI-H+D score of <40% (change from baseline between the first and second cohorts to endpoint). In addition, subjects must have a CGI-S score ≥4 (moderate), NPI-C Delusion domain score of ≥2 on two of the eight items, and NPI Agitation/Aggression domain score ≥4. This embodiment resembles the open-label portion of Example 2 relapse study, but is instead double blind to look for acute benefits on psychosis (resembling the clinical design of EMERGENT-1).

The preceding description is given for clearness of understanding only. No unnecessary limitations should be understood from there, as modifications within the disclosure scope may be apparent to those having ordinary skill in the art. Throughout the specification, where compositions are described as including components or materials, it is contemplated that the compositions can also consist essentially of, or consist of, any combination of the recited components or materials, unless described otherwise. Likewise, where methods are described as including steps, it is contemplated that the methods can also consist essentially of, or consist of, any combination of the recited steps, unless described otherwise. The disclosure illustratively disclosed herein suitably may be practiced in the absence of any element or step which is not specifically disclosed herein.

The practice of a method disclosed herein, and individual steps thereof, can be performed manually and/or with the aid of automation provided by electronic equipment. Although processes have been described regarding embodiments, a person of ordinary skill in the art will readily appreciate that other ways of performing the acts associated with the methods may be used. For example, the order of various of the steps may be changed without departing from the scope or spirit of the method unless described otherwise. Some of the individual steps can also be combined, omitted, or further subdivided into additional steps.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables contained within the generic chemical formulae described herein are specifically embraced by the present invention just as if each combination was individually explicitly recited, to the extent that such combinations embrace stable compounds (i.e., compounds that can be isolated, characterized and tested for biological activity). Also, all subcombinations of the chemical groups listed in the embodiments describing such variables, as well as all subcombinations of uses and medical indications described herein, are also specifically embraced by the present invention just as if each and every subcombination of chemical groups and subcombination of uses and medical indications was individually and explicitly recited herein.

All patents, publications, and references cited herein are hereby fully incorporated by reference. In case of conflict between the present disclosure and incorporated patents, publications, and references, the present disclosure should control.

1-75. (canceled)

76. An oral pharmaceutical composition comprising 20 mg to 66.7 mg xanomeline or a pharmaceutically acceptable salt thereof, and 2 mg to 6.67 mg trospium chloride, wherein the ratio of xanomeline free base to trospium chloride is 10:1.

77. The oral pharmaceutical composition of claim 76, wherein the oral pharmaceutical composition comprises:
20 mg xanomeline or a pharmaceutically acceptable salt thereof, and 2 mg trospium chloride;
30 mg xanomeline or a pharmaceutically acceptable salt thereof, and 3 mg trospium chloride;
40 mg xanomeline or a pharmaceutically acceptable salt thereof, and 4 mg trospium chloride;
50 mg xanomeline or a pharmaceutically acceptable salt thereof, and 5 mg trospium chloride; or
66.7 mg xanomeline or a pharmaceutically acceptable salt thereof, and 6.67 mg trospium chloride.

78. The oral pharmaceutical composition of claim 77, wherein the oral pharmaceutical composition provides an in vivo plasma profile comprising a median Tₘₐₓ for xanomeline of 2 hours and a median Tₘₐₓ for trospium of 1 hour when administered to a patient in need thereof.

79. The oral pharmaceutical composition of claim 78, wherein the xanomeline or a pharmaceutically acceptable salt thereof is formulated as a first component, and the trospium chloride is formulated as a second component.

80. The oral pharmaceutical composition of claim 79, wherein the oral pharmaceutical composition further comprises 0.1% to 0.25% 3-[(4-hexyloxy)-1,2,5-thiadizaol-3-yl]-5-hydroxyl-1-methylpyridin-1-ium.

81. The oral pharmaceutical composition of claim 80, wherein the oral pharmaceutical composition comprises less than 0.5 wt. % of 3-[(4-hexyloxy)-1,2,5-thiadizaol-3-yl]-5-hydroxyl-1-methylpyridin-1-ium after the oral pharmaceutical composition is stored for at least 3 months at 40° C. and 75% relative humidity.

82. The oral pharmaceutical composition of claim 79, wherein the first component is a first plurality of beads, and the second component is a second plurality of beads.

83. The oral pharmaceutical composition of claim 82, wherein the oral pharmaceutical composition is a capsule and the xanomeline is xanomeline tartrate.

84. A method of treating dementia-related psychosis, the method comprising administering the oral pharmaceutical composition of claim 76 three times during a 24-hour period to a patient in need thereof.

85. The method of claim 84, wherein the oral pharmaceutical composition comprises 20 mg xanomeline or a pharmaceutically acceptable salt thereof and 2 mg trospium chloride.

86. The method of claim 85, wherein the oral pharmaceutical composition is a capsule and the xanomeline is xanomeline tartrate.

87. The method of claim 85, wherein the dementia-related psychosis is due to Alzheimer's disease.

88. The method of claim 84, wherein the oral pharmaceutical composition comprises 30 mg xanomeline or a pharmaceutically acceptable salt thereof and 3 mg trospium chloride.

89. The method of claim 88, wherein the oral pharmaceutical composition is a capsule and the xanomeline is xanomeline tartrate.

90. The method of claim 88, wherein the dementia-related psychosis is due to Alzheimer's disease.

91. The method of claim 84, wherein the oral pharmaceutical composition comprises 40 mg xanomeline or a pharmaceutically acceptable salt thereof and 4 mg trospium chloride.

92. The method of claim 91, wherein the oral pharmaceutical composition is a capsule and the xanomeline is xanomeline tartrate.

93. The method of claim 91, wherein the dementia-related psychosis is due to Alzheimer's disease.

94. The method of claim 84, wherein the oral pharmaceutical composition comprises 50 mg xanomeline or a pharmaceutically acceptable salt thereof and 5 mg trospium chloride.

95. The method of claim 94, wherein the oral pharmaceutical composition is a capsule and the xanomeline is xanomeline tartrate.

96. The method of claim 94, wherein the dementia-related psychosis is due to Alzheimer's disease.

97. The method of claim 84, wherein the oral pharmaceutical composition comprises 66.7 mg xanomeline or a pharmaceutically acceptable salt thereof and 6.67 mg trospium chloride.

98. The method of claim 97, wherein the oral pharmaceutical composition is a capsule and the xanomeline is xanomeline tartrate.

99. The method of claim 97, wherein the dementia-related psychosis is due to Alzheimer's disease.
